(19) United States
(12) Patent Application Publication
(10) Pub. No.: US 2013/0338498 A1
(43) Pub. Date: Dec. 19, 2013

(54) Catheter for Intravascular Ultrasound and Photoacoustic Imaging

(71) Applicant: Board of Regents, The University of Texas System, Austin, TX (US)

(72) Inventors: Stanislav Emelianov, Austin, TX (US); Bo Wang, Austin, TX (US); Andrei Karpiouk, Austin, TX (US); Douglas Yeager, Austin, TX (US)

(73) Assignee: Board of Regents, The University of Texas System, Austin, TX (US)

(21) Appl. No.: 13/955,986

(22) Filed: Jul. 31, 2013

Related U.S. Application Data
(63) Continuation-in-part of application No. 13/505,345, filed on Jul. 10, 2012.
(60) Provisional application No. 61/257,390, filed on Nov. 2, 2009.

(51) Int. Cl. A61B 8/12

(52) U.S. Cl. CPC A61B 8/12
USPC 600/431; 600/439

(57) ABSTRACT

The present invention includes a method of imaging and treating a target tissue without the need to occlude or dilute luminal blood in a subject by a combination of intravascular ultrasound and photoacoustic imaging by irradiating the target tissue with electromagnetic radiation at a single wavelength.

### CROSS-REFERENCE TO RELATED APPLICATIONS

This application is a Continuation-in-Part application of U.S. patent application Ser. No. 13/505,345, filed Jul. 10, 2012, which is a 35 U.S.C. 371 National Stage application of International Application No. PCT/US2010/055006, filed Nov. 1, 2010, which claims the benefit of U.S. Provisional Application No. 61/257,390, filed Nov. 2, 2009. The contents of each of which are incorporated by reference in their entirety.

### STATEMENT OF FEDERALLY FUNDED RESEARCH

This invention was made with U.S. Government support by the NIH grant number HL096981. The government has certain rights in this invention.

### TECHNICAL FIELD OF THE INVENTION

The present invention relates in general to the field of combined intravascular ultrasound, photoacoustic and elasticity imaging and intravascular radiation and/or acoustic therapy, and more particularly, to the design and fabrication of an intravascular catheter for combined intravascular ultrasound, photoacoustic and elasticity imaging and for intravascular radiation and/or acoustic therapy.

### BACKGROUND ART

Without limiting the scope of the invention, its background is described in connection with the design and fabrication of an intravascular catheter that combines intravascular ultrasound, photoacoustic and elasticity imaging and is capable of intravascular radiation and/or acoustic therapy.

International Patent Application Publication No. WO/2010/080776 (Thornton, 2010) describes a catheter assembly for an intravascular ultrasound system that includes a catheter and an imaging core disposed in the catheter. The imaging core includes a rotatable driveshaft, at least one light source, and at least one transducer. The at least one light source is disposed at a distal end of the rotatable driveshaft. The at least one light source is configured and arranged for rotating with the driveshaft and also for transforming applied electrical signals to light for illuminating an object in proximity to the catheter. The at least one transducer is also disposed at the distal end of the rotatable driveshaft. The at least one transducer is configured and arranged for rotating with the driveshaft. The at least one transducer is configured and arranged for receiving acoustic signals generated by the object in response to illumination of the object by the light emitted from the at least one light source.

U.S. Pat. No. 7,711,413 issued to Feldman et al., (2010) relates to a catheter imaging probe for a patient. The probe of the Feldman patent includes a conduit through which energy is transmitted. The probe includes a first portion through which the conduit extends. The probe includes a second portion which rotates relative to the conduit to redirect the energy from the conduit. A method for imaging a patient. The method includes the steps of inserting a catheter into the patient. There is the step of rotating a second portion of the catheter relative to a conduit extending through a first portion of the catheter, which redirects the energy transmitted through the conduit to the patient and receives the energy reflected back to the second portion from the patient and redirects the reflected energy to the conduit.

Intravascular ultrasound (IVUS) imaging is widely used to image the atherosclerotic plaques in coronary arteries.¹⁻³ This invasive catheter-based approach is suitable to detect unrecognized disease, lesions of uncertain severity (40% to 75% stenosis), and risk of stratification of atherosclerotic lesions in interventional practice. Histopathalogical information, obtained from the IVUS, is not enough to characterize the plaques due to poor contrast between tissue's ultrasound properties, therefore an additional modality such as intravascular photoacoustic imaging (IVPA) must be used to assess the vulnerability of the plaques.

The IVPA imaging as a part of combined IVUS/IVPA imaging that was demonstrated by Sethuraman et al.⁴ Photoacoustic imaging relies on contrast of light absorption constituents presented inside the arterial tissues and is based on an excitation of a tissue with shot laser pulses with consequent detection of acoustic transients, generated as a result of thermal expansion.⁵⁻⁷ Currently, the photoacoustic imaging is successfully used in different biomedical areas.

The intravascular elasticity imaging as a part of the described intravascular imaging is used to image a distribution of shear elastic modulus in the artery.⁸⁻¹¹ The elasticity imaging relies on a stiffness contrast of artery tissues and plaques content and is based on obtaining several ultrasound images of the same cross-section of the artery during the deformation of the artery's wall under either externally applied force or as a result of normal cardiac cycles or a combination thereof. Using inverse problem formulations, the elasticity distribution is evaluated based on a distribution of the strain tensor components. The elasticity imaging approach is widely used in various clinical applications.¹²⁻¹⁶

Once pathology is detected and its vulnerability is assessed, the same integrated IVUS/IVPA imaging catheter can be used for thermal and/or radiation and/or acoustic therapy of the pathology. In such therapy, the absorbed light energy or acoustic energy or both is converted into a heat leading to necrosis of the pathology tissues. While the pulsed laser is coupled with the catheter to perform diagnostics imaging, the continuous wave (CW) source of a radiation, for instance, a CW laser, should be coupled with the catheter.¹⁷⁻²⁰ The laser is operated at a wavelength that is primarily absorbed by a typical pathology of the cells and molecules.

To enhance the radiation therapy effect, Shah et al. has proposed to use nanoparticles-based contrast agents.¹⁹ Such contrast agents are conjugated with antibodies and can be injected into a blood vessel. After a certain time needed for contrast agents to reach the pathology and label the specific cells, the tissue is irradiated with CW laser light. The radiation is primarily absorbed by nanoparticles which cause heating. The heated nanoparticles lead to a temperature increase in the tissue environment thus inducing therapeutic effects.

In the acoustic therapy, a relatively low-frequency, high-intensity focused ultrasound (HIFU) beam is directed in the area of the detected pathology and, due to acoustic absorption, scattering and/or reflecting, leads to a temperature increase thus resulting in necrosis of the pathology tissues.^{21,22} The HIFU treatment is also well-known modality of non-invasive therapy and can be performed either from outside or from the inside of the artery. However, to perform all of these imaging and therapy procedures clinically, specially designed catheters need to be used. Currently available catheters cannot be used both for combined IVUS, IVPA and elasticity imaging and for radiation and/or acoustic therapy.

The present invention describes two representative designs of fiber-based integrated catheters both for combined IVUS/IVPA imaging and for intravascular radiation and/or acoustic therapy. One design is based on single-element catheter-based ultrasound transducers coupled with specially designed light delivery systems. In this approach, the light delivery system is based on the side fire fiber, similar to that utilized for biomedical optical spectroscopy²³ or on the micro-optics in a manner of a probe for optical coherent tomography. In the second design, the integrated catheter is based on ultrasound array transducer that also is coupled with the side fire fiber or micro-optics light delivery system. In both types of the integrated catheters, the light delivery systems were designed to direct the light into the area or tissues imaged by the ultrasound transducer. In addition to that, the CW radiation utilized for radiation therapy is also delivered in the same area. Finally, an intravascular acoustic therapy can be performed using one or more ultrasound units that deliver the acoustic radiation in the desired area of the artery. Tunable in wide spectral range a Ns-pulsed laser-based system was employed as a light source for photoacoustic imaging, while ultrasound pulser/receiver was used for ultrasound imaging.

### SUMMARY OF THE INVENTION

In another embodiment the present invention provides a method of imaging and treating a target tissue in a subject comprising the steps of: (i) identifying a subject in need of treatment of a target tissue using an intravascular imaging and therapeutic device capable of combined intravascular ultrasound, photoacoustic and elasticity imaging, (ii) irradiating the target tissue with radiation and/or ultrasound energy from an intravascular imaging and therapeutic device comprising: one or more intravascular ultrasound imaging and therapeutic units comprising a proximal end and a distal end, wherein the distal end comprises one or more single-element ultrasound transducers, one or more ultrasound arrays or a combinations thereof, wherein the proximal end comprises a port connecting at least one ultrasound unit to a pulser/reliever; one or more optical units comprising a proximal end and a distal end combination, wherein the distal end comprises one or more optical fibers, one or more optical bundles or a combination of both, wherein the proximal end comprises a port to couple at least one optical unit to a pulsed light source and/or to couple at least one optical unit to a continuous wave (CW) light source wherein a majority of a laser and ultrasound energy is Omni-directionally directed at a target tissue; an ultrasound pulser/receiver connected to the proximal end of the one or more ultrasound imaging and therapeutic units; a pulsed light source connected to the proximal end of the one or more optical units having a pulsed laser fluence; a CW light source connected to the proximal end of one or more optical units having a CW laser fluence; and an imager connected to the proximal end of the unit to capture one or more ultrasound, optical and elasticity images, and the imager is capable both of reconstruction of distributions of an ultrasound impedance, a shear elastic modulus and an optical absorption in an imaged target tissue cross-section and of performing a radiation and/or an acoustic therapy, (iii) reconstructing a distribution of an ultrasound impedance, a distribution of a shear elastic modulus and a distribution of an optical absorption in an imaged tissue cross-section or a combination of thereof, (iv) performing an acoustic and/or a radiation therapy of the target tissues, (v) performing the imaging and therapy all together or separately in any combinations thereof.

In one aspect of the method of the present invention related to imaging the distribution of the ultrasound impedance is reconstructed by transmitting of short ultrasound waves into the target tissue with consequent detection of reflected and scattered ultrasound waves. In another aspect of the method of the present invention the distribution of the optical absorption is reconstructed by transmitting of short light pulses into the target tissue with a consequent detection of ultrasound waves generated in the tissue due to thermal expansion by the irradiated light. In yet another aspect the distribution of shear elastic modulus is reconstructed by collecting of multiple ultrasound images where one or more strain tensor components are measured assessing local tissue's displacement in response to an external or a cardiac loading.

In one aspect of the method of the present invention related to the therapy the one or more ultrasound units irradiate an artery with long ultrasound pulses to perform an acoustic therapy of the artery. In another aspect the one or more optical units can irradiate tissues by CW or long light pulses to perform an optical therapy. In yet another aspect the optical and the acoustic therapy can be performed either simultaneously or separately. In a related aspect the reconstruction of the distributions and therapy can be performed either simultaneously or consequently, where ultrasound, photoacoustic and elasticity imaging can be performed during the therapy using optical and ultrasound units that are not engaged in therapy to guide and monitor the treatment. In a certain aspect the imager is capable of providing an imaging result or a therapy result in a format determined by a user.

In one embodiment, the present invention includes a method of imaging and treating a target tissue without the need to occlude or dilute luminal blood in a subject comprising the steps of: identifying a subject in need of treatment of a target tissue using an intravascular imaging and therapeutic device capable of combined intravascular ultrasound and photoacoustic imaging; irradiating the target tissue with electromagnetic radiation at a single wavelength from an intravascular imaging and therapeutic device comprising: a catheter with a proximal end and a distal end; one or more intravascular ultrasound units comprising a proximal end and a distal end, wherein the distal end comprises one or more single-element ultrasound transducer elements at a proximal end; one or more optical units comprising a proximal end and a distal end combination, wherein the distal end comprises at least one of one or more optical fibers or one or more optical bundles, wherein the proximal end comprises a port to couple at least one optical unit to a light source that irradiates at a single wavelength; an imager connected to the proximal end of the unit to capture one or more ultrasound and optical images; and performing at least one of an imaging or a therapy of the target tissue. In one aspect, the single wavelength is selected from one of 1060, 1064, 1070, 1200, 1210, 1700, 1710, 1715, 1720, 1725, 1730, or 1740 nm. In another aspect, the distribution of the ultrasound impedance is reconstructed by transmitting of short ultrasound waves into the target tissue with consequent detection of at least one of reflected or scattered ultrasound waves. In another aspect, the distribution of the optical absorption is reconstructed by transmitting of short light pulses of the same wavelength into the target tissue with a consequent detection of ultrasound waves generated in the tissue due to thermal expansion of the tissue due to absorbed light energy. In another aspect, the one or more ultrasound units irradiate an artery with long ultrasound pulses to perform an acoustic therapy of the artery. In another aspect, the one or more optical units can irradiate tissues by CW or long-pulse light to perform an optical therapy. In another aspect, the at least one of optical therapy, acoustic therapy, tissue target imaging, or therapy and imaging can be performed either simultaneously or separately. In another aspect, the imager is capable of providing imaging results or therapy results in a format determined by a user. In another aspect, the imager has a penetration depth of 1 to 5 mm. In another aspect, the imager has a resolution of 50 micrometers. In another aspect, the imager is able to detect the presence and spatially resolved location of lipid in the arterial wall. In another aspect, the imager modifies the contrast between lipid tissue and water-based tissue by increasing the temperature at the target tissue, wherein photoacoustic signal intensity of the lipid based tissue decreases and the water-based tissue increases. In another aspect, the method further comprises the step of mechanically or electrically steering an ultrasound beam to generate a two-dimensional cross-sectional image of the target. In another aspect, the method further comprises the step of using a contrast agent in the target tissue.

In another embodiment, the present invention includes a method of imaging and treating a target tissue in vivo without the need to occlude or dilute luminal blood in a subject comprising the steps of: identifying a subject in need of treatment of a target tissue using an intravascular imaging and therapeutic device capable of combined intravascular ultrasound and photoacoustic imaging; irradiating the target tissue with electromagnetic radiation at a single wavelength selected from wavelengths in which lipid and water have a different index of refraction comprising: a catheter with a proximal end and a distal end; one or more intravascular ultrasound units comprising a proximal end and a distal end, wherein the distal end comprises one or more single-element ultrasound transducer elements at a proximal end; one or more optical units comprising a proximal end and a distal end combination, wherein the distal end comprises at least one of one or more optical fibers or one or more optical bundles, wherein the proximal end comprises a port to couple at least one optical unit to a light source that irradiates at a single wavelength; an imager connected to the proximal end of the unit to capture one or more ultrasound and optical images; and performing at least one of an imaging or a therapy of the target tissue in vivo. In one aspect, the single wavelength is selected from one of 1060, 1064, 1070, 1200, 1210, 1700, 1710, 1715, 1720, 1725, 1730, or 1740 nm. In another aspect, the distribution of the ultrasound impedance is reconstructed by transmitting of short ultrasound waves into the target tissue with consequent detection of at least one of reflected or scattered ultrasound waves. In another aspect, the distribution of the optical absorption is reconstructed by transmitting of short light pulses of the same wavelength into the target tissue with a consequent detection of ultrasound waves generated in the tissue due to thermal expansion of the tissue due to absorbed light energy. In another aspect, the one or more ultrasound units irradiate an artery with long ultrasound pulses to perform an acoustic therapy of the artery. In another aspect, the one or more optical units can irradiate tissues by CW or long-pulse light to perform an optical therapy. In another aspect, the at least one of optical therapy, acoustic therapy, tissue target imaging, or therapy and imaging can be performed either simultaneously or separately. In another aspect, the imager is capable of providing imaging results or therapy results in a format determined by a user. In another aspect, the imager has a penetration depth of 1 to 5 mm. In another aspect, the imager has a resolution of 50 micrometers. In another aspect, the imager is able to detect the presence and spatially resolved location of lipid in the arterial wall. In another aspect, the imager modifies the contrast between lipid tissue and water-based tissue by increasing the temperature at the target tissue, wherein photoacoustic signal intensity of the lipid based tissue decreases and the water-based tissue increases. In another aspect, the method further comprises the step of mechanically or electrically steering an ultrasound beam to generate a two-dimensional cross-sectional image of the target. In another aspect, the method further comprises the step of using a contrast agent in the target tissue.

### BRIEF DESCRIPTION OF THE DRAWINGS

For a more complete understanding of the features and advantages of the present invention, reference is now made to the detailed description of the invention along with the accompanying figures and in which:

FIGS. 1A to 1C are representations of the side fire fiber-based integrated IVUS/IVPA imaging catheter: FIG. 1A demonstrates the operation of the catheter. If total internal reflection (TIR) effect is reached for the light propagating along the fiber's axis, this light will be reflected from the polished surface (beam 1). Due to non-zero numerical aperture (NA) of the fiber and with the same β, TIR conditions are all the more valid for some portion of light propagating at the angle 0 to α in respect to the fiber axis (beam 2). However, under the same conditions, loses of the light are possible since TIR effect is not valid for portion of light propagating at the angle 0 to −α in respect to the fiber axis (beam 3), FIG. 1B is a photograph of the proximal end of the integrated IVUS/IVPA side fire fiber-based catheter utilizing the TIR effect, and FIG. 1C depicts an alignment between ultrasound and light beams;

FIGS. 2A and 2B show a mirror-based integrated IVUS/IVPA catheter: FIG. 2A is a photograph of distal end of the mirror-based integrated IVUS/IVPA imaging catheter and FIG. 2B depicts an alignment of the ultrasound and light beams;

FIGS. 3A and 3B shows a distal end of a side fire fiber-based IVUS/IVPA imaging catheter capable of rotating inside a lumen: FIG. 3A shows a photograph of the view of the catheter and a magnified view of an ultrasound transducer and an outlet of light delivery system are shown and FIG. 3B is a schematic diagram of the catheter shown to clarify its construction;

FIG. 4 demonstrates designs of a distal end of the integrated IVUS/IVPA imaging catheters—a schematic diagram demonstrating a design wherein an ultrasound unit is a single-element market-available intravascular ultrasound imaging catheter that rotates inside of a lumen while optical unit comprises several optical fibers with a light delivery system installed at each optical fiber. The light delivery system is stationary and irradiates a whole cross-section of an artery imaged by ultrasound imaging catheter;

FIGS. 5A and 5B show a distal end of a ultrasound array-based integrated IVUS/IVPA catheter with light delivery system that utilizes a single side fire fiber: FIG. 5A is a schematic diagram demonstrating a design wherein an ultrasound unit is an ultrasound array that is not rotated while the optical unit is a single optical fiber with installed light delivery system. The optical fiber is rotated inside the lumen and consequently irradiates parts of an artery that is been imaged by the ultrasound array, FIG. 5B shows a photograph of the prototype of the integrated IVUS/IVPA catheter based on an ultrasound array and light delivery system utilizing TIR effect;

FIG. 6A is a photograph and FIG. 6B is a diagram of the pencil rod-based phantom used in the IVUS/IVPA tissue-mimicking studies;

FIG. 7 shows a block diagram of the combined IVUS/IVPA imaging system operating with an integrated catheter;

FIGS. 8A-8D show study images obtained by the side fire fiber- and mirror-based integrated IVUS/IVPA catheter: FIG. 8A is ultrasound, FIG. 8B is a photoacoustic image of the phantom without tissue-mimicking environment in water obtained using the side fire fiber-based integrated IVUS/IVPA catheter, FIG. 8C is ultrasound, and FIG. 8D is a photoacoustic image of the phantom without tissue-mimicking environment in water obtained using the mirror-based integrated IVUS/IVPA catheter;

FIGS. 9A-9D show study images obtained by the side fire fiber- and mirror-based integrated IVUS/IVPA catheter: FIG. 9A is ultrasound, FIG. 9B is a photoacoustic image of the phantom with tissue-mimicking environment in water obtained using the side fire fiber-based integrated IVUS/IVPA catheter, FIG. 9C is ultrasound, and FIG. 9D is a photoacoustic image of the phantom with tissue-mimicking environment in water obtained using the mirror-based integrated IVUS/IVPA catheter;

FIGS. 10A and 10B show study images obtained by the side fire fiber-integrated IVUS/IVPA catheter: FIG. 10A is ultrasound and FIG. 10B is a photoacoustic image of the phantom without tissue-mimicking environment in 20% solution of low-fat milk obtained using the side fire fiber-based integrated IVUS/IVPA catheter;

FIGS. 11A and 11B show study images obtained by the mirror-based integrated IVUS/IVPA catheter: FIG. 11A is an ultrasound image and FIG. 11B is a photoacoustic image of the phantom with tissue-mimicking environment in water obtained using the mirror-based integrated IVUS/IVPA catheter. The catheter is realigned such that transducer is shifted two millimeters away from the mirror;

FIG. 12A demonstrates the experimental setup where the phantom in a plastic mold was stored in a water tank while the catheter is inserted into the phantom lumen and rotated within;

FIGS. 12B and 12C shows study images obtained by the rotatable side fire fiber-based integrated IVUS/IVPA catheter: FIG. 12B is an ultrasound, and FIG. 12C is a photoacoustic image of the phantom with tissue-mimicking environment obtained using the mirror-based integrated IVUS/IVPA catheter;

FIGS. 13A and 13B show study images obtained using the ultrasound array-based integrated IVUS/IVPA catheter with side fire fiber-based light delivery system (FIG. 5B): FIG. 13A is ultrasound and FIG. 13B is a photoacoustic image of the phantom without tissue-mimicking environment in water obtained using the ultrasound array-based integrated IVUS/IVPA catheter with side fire fiber-based light delivery system irradiating the one pencil rod;

FIGS. 14A to 14C shows combined intravascular photoacoustic (IVPA)/intravascular ultrasound (IVUS) imaging system used in in vivo and ex vivo studies. FIG. 14A shows a block diagram of the combined IVPA/IVUS imaging system. FIG. 14B shows as X-ray image of the WHHL rabbit with an integrated IVPA/IVUS imaging catheter advanced into the abdominal aorta. FIG. 14C shows a photograph of the distal end of the integrated IVPA/IVUS imaging catheter. The catheter includes a single element IVUS imaging catheter and side-fire optical fiber;

FIGS. 15A to 15C shows in vivo intravascular photoacoustic (IVPA)/intravascular ultrasound (IVUS) imaging of lipid in the abdominal rabbit aorta. FIG. 15A shows IVPA image shows the spatially resolved distribution of lipid deposits inside the vessel. FIG. 15B shows IVUS image shows the anatomy of the vessel wall. IVPA and IVUS images are displayed using 20 dB and 35 dB display dynamic ranges, respectively. FIG. 15C shows the location of lipid deposits in the vessel wall can be visualized in combined IVPA/IVUS image. Lipid deposits are mainly located in the intimal layer of the artery;

FIGS. 16A to 16H shows ex vivo intravascular photoacoustic (IVPA)/intravascular ultrasound (IVUS) imaging of lipid in the atherosclerotic rabbit aorta. FIGS. 16A to C show IVPA, IVUS and combined IVPA/IVUS images of the rabbit aorta acquired ex vivo in the human RBCs solution. The vessel wall can be barely visualized in the IVUS image because of the strong acoustic scattering from stationary luminal blood. FIGS. 16E to 16G show IVPA, IVUS and combined IVPA/IVUS images of the same cross-section of the vessel containing diluted RBCs solution. IVPA imaging depth is increased due to the reduced attenuation of light in the luminal blood. IVPA and IVUS images are displayed using 25 dB and 35 dB display dynamic ranges, respectively. FIGS. 16D and 16H show H&E and Oil red O stains, respectively, confirmed lipid deposits in the intimal layer of the aorta; and

FIGS. 17A to 17F show ex vivo intravascular photoacoustic (IVPA)/intravascular ultrasound (IVUS) imaging of lipid core inside a human right coronary artery (RCA). FIG. 17A IVPA image of the formalin fixed human RCA. Strong IVPA signals from lipid deposits can be observed at the 3-6 o'clock position. FIG. 17B shows IVUS image showed the luminal blood and vessel wall. FIG. 17C shows combined IVPA/IVUS image shows that the lipid region is deep inside the intimal layer of the vessel. FIGS. 17D and 17E show 32 and 34 H&E stain of the imaged cross-section showed lipid core (pointed by an arrow in 17E) in the thickened intimal layer of the vessel wall. FIG. 17F shows CD68 stain confirmed that the lipid core (pointed by an arrow) is rich in macrophages. An intimal artifact occurred during section of the specimen (3 o'clock in FIG. 17D to 17F).

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

The term “photoacoustic or optoacoustic imaging” as used herein applies to any imaging method in which an electromagnetic radiation generates a detectable pressure wave or sound from which an image is calculated. As used herein, the term “intravascular” refers to within a blood vessel (for example, an artery, vein or capillary).

As used herein the term “catheter” broadly encompasses a wide array of devices for accessing remote locations, particularly within interior bodily vessels and cavities. Medical catheters may be used for tissue sampling, temperature measurements, drug administration or electrical stimulation to a selected tissue. With fiber optics, they may carry light for visual inspection of tissues. Medical catheters are generally maneuverable through anatomical cavities, vessels, and other structures of the body.

The term “optical fiber” as used herein is generally understood to refer to a light wave guide which, in its simplest form, consists of at least two layers of glass. One layer forms the core of the fiber and the other forms the fiber cladding and is placed round the core, whilst having a refractive index below that of the core.

The term “transducer array” as used herein refers to a series made up of a plurality of ultrasonic transducers, preferably situated directly adjacent to one another. The individual transducers are preferably positioned in alignment and generate, for example, flat or cylindrical ultrasonic waves. However, the transducer array may also be designed in such a way that spherical, ellipsoidal or otherwise curved wave fronts are generated.

The term “laser fluence” as used in the specification is a unit indicating the energy density of laser-light, which is obtained by integrating the energy amount per unit area by time. To be more specific, the “laser fluence” is an average intensity of laser light measured at a laser source or in an irradiation region.

The term “total internal reflection” refers to the reflection that occurs within a substance because the angle of incidence of light striking a boundary surface is in excess of the critical angle. The term “angle of incidence” refers to the angle formed between a ray of light striking a surface and the normal to the surface at the point of incidence. A “light ray” or “ray of light” is one of the radii of a wave of light that indicates the direction of light travel.

The term “image” as used herein broadly refers to any multidimensional representation, whether in tangible or otherwise perceptible form or in a computer memory or a storage medium, whereby a value of some characteristic is associated with each of a plurality of locations corresponding to dimensional coordinates of an object in physical space, though not necessarily mapped one-to-one thereon. The term “image” also includes an ordered representation of detector signals corresponding to spatial positions. For example, the image may be an array of values within an electronic memory or holographic medium, or, alternatively, a visual image may be formed on a display device such as a video screen or printer. Thus, for example, the graphic display of the spatial distribution of some feature, such as atomic number, in one or more colors constitutes an image. Similarly, “imaging” refers to the rendering of a stated physical characteristic in terms of one or more images.

The term “proximal end” is considered to be the end closest to an operator, while the term “distal end” indicates the end of the device farthest from the operator.

The term “micro-optics” includes fine structures causing refraction and/or diffraction, the structures having characteristic depths/heights and often also widths of typically a few micrometers, for example of 0.5 μm-200 μm, preferably of between 1 μm and about 50 μm or between 1 μm and about 30 μm. In other words, the characteristic profile depths and the profile widths are of the order of a few wavelengths up to a few tens of wavelengths for refractive optics and of about one wavelength up to a few wavelengths for diffractive optics.

The term “wavelength” as used herein refers to the actual physical length comprising one full period of electromagnetic oscillation of a light ray or light beam. The term “irradiation” is broadly defined herein to include any process for treating or exposing something to light or other radiant energy to create a relatively more visibly marked portion compared to surrounding portions. The term “single wavelength” refers to the use of a single wavelength of light at a time, or wherein the device only produces a single wavelength, or is filtered or otherwise restricted to a single wavelength. The skilled artisan will recognize that any given wavelength will have some variability with any device while still maintaining its operation as a single wavelength of light. The present invention can be used with or without contrast agents. Non-limiting examples of single wavelengths that can be used with the present invention include 1060, 1064, 1070, 1200, 1210, 1700, 1710, 1715, 1720, 1725, 1730, or 1740 nm.

### Example 1

The present invention describes two designs of an integrated intravascular ultrasound, photoacoustic (IVUS/IVPA) and elasticity imaging catheter capable both of combined intravascular ultrasound, photoacoustic, and elasticity imaging and of radiation and/or acoustic therapy on an artery and/or nearby tissues. Such catheter include of one or more ultrasound units that are either a single element ultrasound transducer or an ultrasound array transducer or a combination thereof, and one or more optical units that comprise one or more optical fibers, one or more optical bundles or a combination thereof. A light delivery system is mounted on one or more optical units. The one or more ultrasound units and one or more optical units are assembled into a single device such that ultrasound and optical beams propagate orthogonally to the longitudinal axis of the catheter with maximum overlap with each other.

The elasticity imaging of an artery is performed by one or more ultrasound units and is based on an intravascular ultrasound imaging of the artery. The radiation therapy is performed by one or more optical units that are also utilized in intravascular photoacoustic imaging. The acoustic therapy is performed by one or more ultrasound units that are also utilized in both ultrasound and photoacoustic imaging. The radiation therapy is performed by one or more optical units that are also utilized in photoacoustic imaging. Therefore, if the integrated IVUS/IVPA catheter is capable of combined IVUS/IVPA imaging, then it is also capable both of an intravascular elasticity imaging and of radiation therapy. A detailed description of two designs of an integrated IVUS/IVPA imaging catheter is given below.

A side fire fiber-based and a mirror-based catheter both utilizing a single-element ultrasound transducer and a side fire fiber-based catheter utilizing and ultrasound array are described in the present invention. Commercially available ultrasound transducers are utilized for ultrasound imaging and detection of photoacoustic transients. Laser pulses are delivered by custom-designed optical system mounted on the distal tip of a single optical fiber combined with the ultrasound transducer or transducer array into a single device.

Cardiovascular diseases represent a significant clinical problem with more than a million deaths annually due to problems associated with the arteries. The most common reason of the mortality is the formation and development of atherosclerotic plaques on artery's walls. These plaques narrow the cross-section of the vessels thus obstructing the normal blood flow.²⁷ In addition, the vulnerability of the atherosclerotic plaques depends on their composition.^{28,29} Therefore, a successful treatment of the disease can be achieved if the distribution and the vulnerability of the plaques are diagnosed reliably.

A number of imaging techniques can be applied for diagnostic and treatment of the plaques. IVUS imaging is used to image the atherosclerotic plaques in coronary arteries.¹⁻³ This invasive catheter-based approach can detect unrecognized disease, lesions of uncertain severity (40% to 75% stenosis), and risk of stratification of atherosclerotic lesions in interventional practice. However, histopathalogical information, obtained from the IVUS, is not enough to characterize the plaques due to poor contrast between tissue's ultrasound properties. To further assess the vulnerability of the plaques, the present inventors previously introduced IVPA imaging.

The photoacoustic imaging component of a combined IVUS/IVPA was demonstrated by Sethuraman et al.⁴ A number of scientific groups use the photoacoustic technique successfully for various vascular medical applications.³⁰⁻³⁵ Photoacoustic imaging relies on contrast of light absorption properties of the tissues and is based on an excitation of a tissue with laser pulses and with consequent detection of acoustic transients, generated as a result of thermal expansion.⁵⁻⁷ The applicability of the combined IVUS/IVPA imaging to detect and differentiate atherosclerosis has already been demonstrated,^{36,37} but a specially designed catheter is needed to use such imaging clinically.

For IVUS imaging, an ultrasound imaging unit—a single-element catheter-based ultrasound transducer³⁸ or an ultrasound array³⁹ is used clinically. To realize the IVPA imaging modality, an optical unit based on single optical fiber, optical fiber bundle or a combination thereof should be incorporated with the ultrasound imaging unit. The fiber-based imaging units have already been reported for photoacoustic⁴⁰ and ultrasound⁴¹ imaging separately, however, the integrated IVUS/IVPA imaging device capable both of combined IVUS/IVPA and elasticity imaging and of radiation and/or acoustic therapy has not been realized. In addition, both reported designs are selectively sensitive to signals coming along an axis of the catheter while a selectivity to signals coming across the axis is required for clinical applications.³²

In the present invention, two designs of integrated IVUS/IVPA imaging catheters for combined IVUS/IVPA imaging are described. Particularly, one design is based on single-element catheter-based ultrasound transducers incorporated longitudinally with a single optical fiber. In another design, an ultrasound array is incorporated with a fiber-based optical unit. In both designs, single optical fibers with a proximal end polished flat and perpendicularly to the optical axis of the fiber were used. The specially designed light delivery systems capable of redirecting light at near right angle related to the optical axis of the fiber were mounted on a distal end of the fiber. One design of the light delivery systems uses the side fire fiber, similar to that utilized for biomedical optical spectroscopy.²³ The second design uses the micro-optics like a probe for optical coherent tomography.⁴² All designs of integrated IVUS/IVPA imaging catheters must have a port for guide wire which is not shown in prototypes.

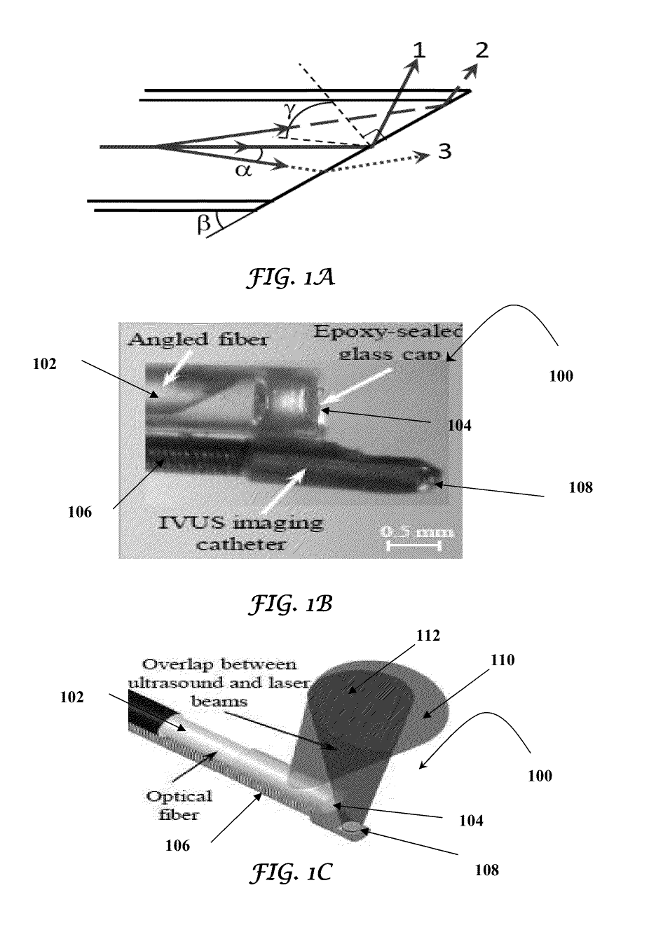

The operation of side fire fiber shown schematically in FIG. 1A is based on the total internal reflection (TIR) effect. The critical angle γ of TIR is defined as:

$$γ = arcsin ( \frac{{n}_{med}}{{n}_{core}} ) , \qquad ( 1 )$$

where n_{med} and n_{core} are refraction coefficients of medium outside of a fiber and of a fiber's core. If β is an angle of fiber's polishing then TIR effect appears when

β≦90−γ (2)

However, the Eq. (2) has to be corrected due to fiber's non-zero NA. A full cone angle 2α of light inside of a fiber is defined as:

$$2 \cdot α = 2 \cdot [ 90 - arcsin ( \frac{{n}_{cl}}{{n}_{core}} ) ] , \qquad ( 3 )$$

where n_{cl} is a refraction coefficient of fiber's cladding. Considering Eqs. (1-3), TIR effect appears when the polishing angle β₀′ obeys the condition:

$${β}_{0}^{′} \leq β + α = 180 - arcsin ( \frac{{n}_{med}}{{n}_{core}} ) - arcsin ( \frac{{n}_{cl}}{{n}_{core}} ) . \qquad ( 4 )$$

A decrease of β results an evaluation of the TIR effect contribution up to 100% when β reaches β₀:

$${β}_{0} \leq β - α = arcsin ( \frac{{n}_{cl}}{{n}_{core}} ) - arcsin ( \frac{{n}_{med}}{{n}_{core}} ) . \qquad ( 5 )$$

Since a near right-angle light rotation is required, the blood in a lumen has to be substituted by a gas near fiber's distal end. In the case of air, angles βₒ′ and β will comprise 62.25° and 31.03° respectively, so light can be redirected within angle of 0 to 62.06° fully and within 62.06° and 124.5° partially.

The photograph of a working prototype of the side fire fiber-based integrated IVUS/IVPA imaging catheter 100 and its schematic diagram are shown in FIGS. 1B and 1C, respectively. In this particular case, the distal end of the optical fiber 102 is polished at the angle of β=33° and an air cup 104 is used as an air trapper near the distal end of the optical fiber 102. The ultrasound transducer 108 is on tube 106 and is fixed face to fiber 102 as it is shown in FIG. 1C using the shrinking tubing in a longitudinal position excluding direct interaction of ultrasound beam 112 with the light delivery system 102 and 104. The light divergence after the catheter was measured to be 26° while angle between light 110 and ultrasound beams 112 was 24°.

In the particular design of the micro-optic-based integrated IVUS/IVPA imaging catheter, the distal end of the optical fiber is polished flat and is perpendicular to the optical axis of the fiber, and a small mirror is used to rotate light. The mirror is attached to the fiber using a custom-made brass fixture comprising of a thin-wall cut along pipe and soldered to the pipe is a bended plate. Since this design does not rely on refraction coefficients of fiber's core and the medium, there is no need to have gas trapping cup near distal end of the light delivery system. However, the protective cup was installed on the distal end of the optical fiber to protect a patient from small sharp parts of the fiber if it would be broken accidentally as well as to make the distal end of the fiber round. To decrease the light losses, distilled water should fill the cup. The resulting angle between optical axis of the fiber and the plate with the glued mirror is chosen for better overlapping of light and ultrasound beams.

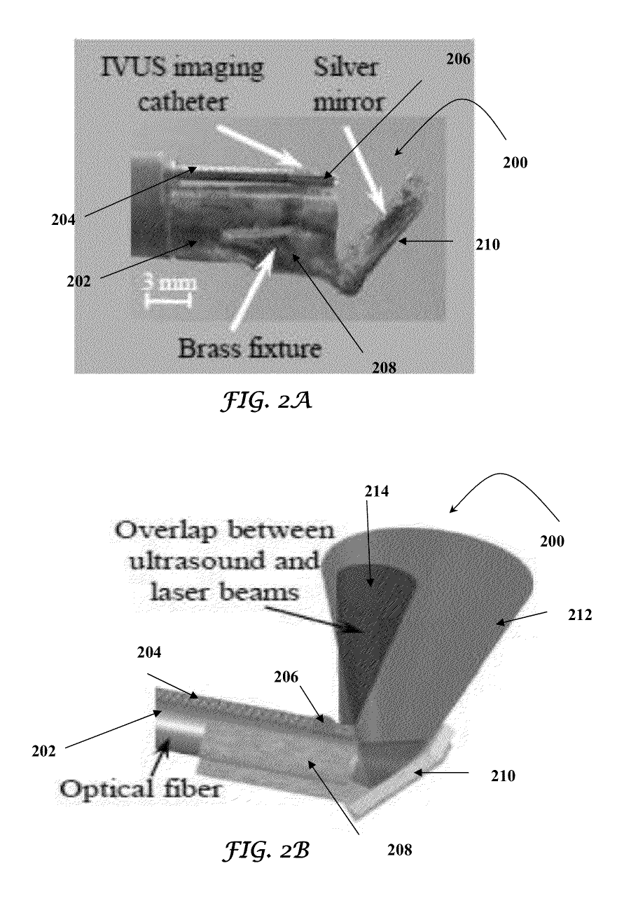

The photograph of mirror-based IVUS/IVPA catheter 200 and its schematic diagram are shown in FIG. 2A and FIG. 2B, respectively. The ultrasound transducer 206 was fixed face from fiber 202 using shrinking tubing 204 in the position resulted the maximum overlap of the ultrasound 214 and light beams 212. The angle between fiber's 202 optical axes and mirror 210 fixed with brass fixture 208 is approximately 52°. An angle between ultrasound and optical axes was estimated to be approximately 14°. Since the optical fiber 202 with NA of 0.39 and core refraction coefficient of 1.457 is located in water, the light divergence of the catheter comprises 17°.

A commercially available IVUS catheter (model Atlantis™SR plus, Boston Scientific SciMed, Inc.) based on a single-element ultrasound transducer with central frequency of 40 MHz was used.³⁸ An outer diameter of the catheter with a 500-μm-diam active element was 1 mm. Side fire fiber-based and mirror-based light delivery systems utilized single multimode optical fibers FT600EMT and FT1500EMT respectively (Thorlabs, Inc.). Laser threshold of silica core material is 1 MW/cm². Proximal ends of both fibers are polished regularly.

In side fire fiber-based light delivery system, the air cup was made out of quartz pipe with inner diameter of 700 μm and outer diameter of 1 mm. The air trapping cup was sealed with an approximately 500 μm layer of epoxy (Devcon, Inc.) and installed on the distal end of the fiber to have a gap between the fiber and the cup behind of the fiber.

In the micro-optic-based light delivery system, small optical parts such as micro-mirrors, micro-lenses, micro-prisms or combinations thereof can be used to redirect light. In this particular example, custom-made micro-mirrors were used as a micro-optics. The mirrors were fabricated by thermal evaporation of silver powder (part #303372-10G, Sigma-Aldrich, Inc.) on 2.5×3-mm pieces of 1-mm thick glass. The laser damage threshold for the mirrors was estimated to be 170 mJ/cm². No protective cup was used in the prototype.

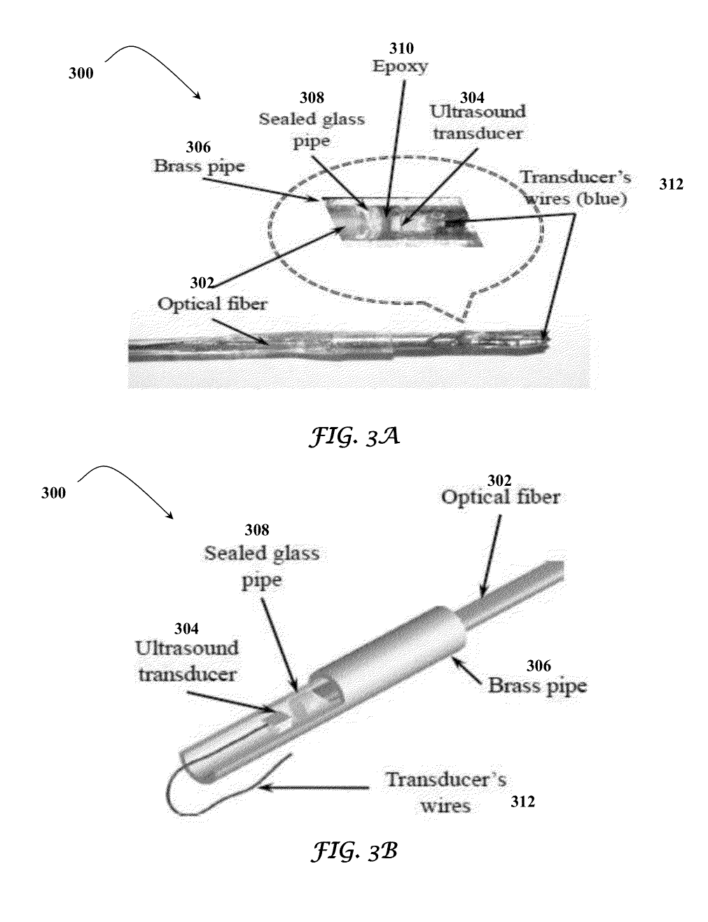

A photograph of an advanced prototype of the side fire fiber-based integrated IVUS/IVPA imaging catheter 300 utilizing a single-element ultrasound transducer 304 is shown in FIG. 3A. The optical fiber 302 an brass pipe 306 with a core diameter of 600 μm and polyamide jacketing (Polymicro Technologies, Inc.) was polished at the angle of 35°. The gas trapping cup 308 with air inside was installed on the distal tip of the fiber. The PVDF-based unfocused ultrasound transducer 304 with the central frequency of 40 MHz capable of working in both ultrasound and photoacoustic modes is incorporated with the light delivery system 302 and 308 using a 10-mm long brass pipe 306 with inner and outer diameters of 1 and 1.5 mm and is connected with transducer wires 312. An epoxy 310 was utilized to fix together all parts. An assembly of the advanced integrated imaging catheter 300 is shown in details in FIG. 3B. Comparing with the previously described prototypes, such integrated IVUS/IVPA imaging catheter 300 is capable of imaging immobile tissues while the catheter itself is rotated inside a lumen.

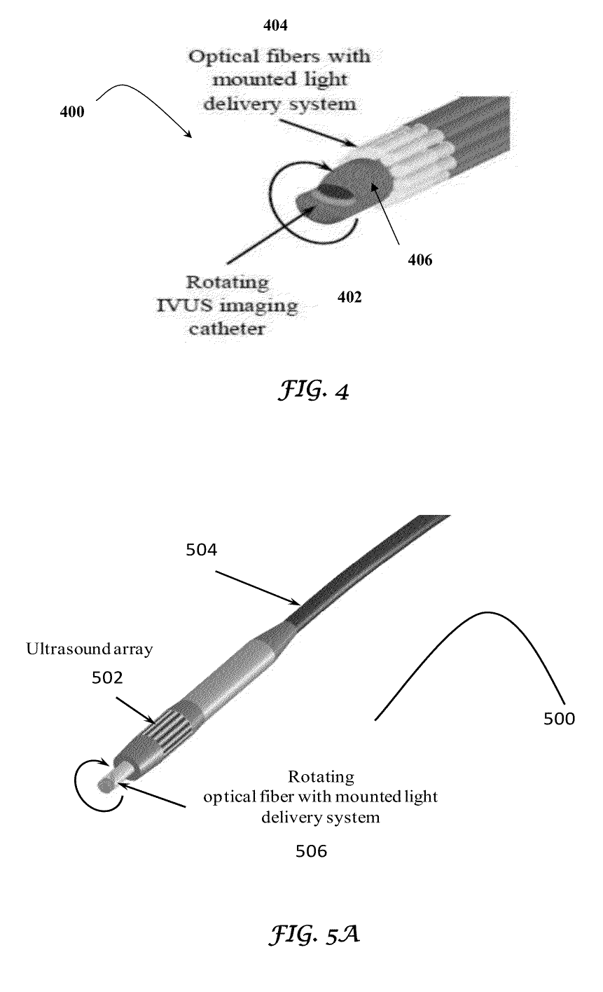

Another possible design of the integrated IVUS/IVPA imaging catheter 400 based on the single element-based IVUS imaging catheter 402 is shown schematically in FIG. 4. In this design, the light delivery system is based on an optical fiber bundle 404 where the optical fibers of the bundle 404 are fixed a rigid cylinder 406 and distributed around the IVUS imaging catheter 402. Either side fire fiber-based or micro-optic-based light delivery system can be installed on the distal tips of each optical fiber 402. The side fire fiber or the micro-optic-based light delivery system directs light away from the ultrasound transducer. The IVUS imaging catheter 402 is rotating inside of this cylinder 406 while the cylinder 406 and fixed on it optical bundle 404 is immobile. Unlike the previously described prototypes, in this design, thinner optical fibers with diameters ranging from 50 μm provides the flexibility that makes the integrated IVUS/IVPA imaging catheter 400 suitable for in vivo imaging.

The schematic diagram design of an integrated IVUS/IVPA imaging catheter 500 based on an ultrasound imaging catheter 504 is shown in FIG. 5A. An ultrasound array 502 is utilized both to probe a tissue in pulse-echo mode and to detect photoacoustic transients generated into the tissue as a result of light-tissue interaction. The side fire fiber-based light delivery system is installed on a single optical fiber 506. The photograph of the prototype of the ultrasound array-based integrated IVUS/IVPA catheter 500 used in the studies is shown in FIG. 5B. The design utilizes the market-available ultrasound array 502 (Eagle Eye, Volcano, Inc.) with the central frequency of 20.75 MHz and the bandwidth of 40% and a single optical fiber 506 with a core diameter of 600 μm in Polyamide jacketing (Polymicro Technologies, Inc.). The mechanical rotation of the optical fiber 506 should be synchronized with the electronic rotation of ultrasound one emitted by the ultrasound array 502. The light delivery system is based on side fire fiber.

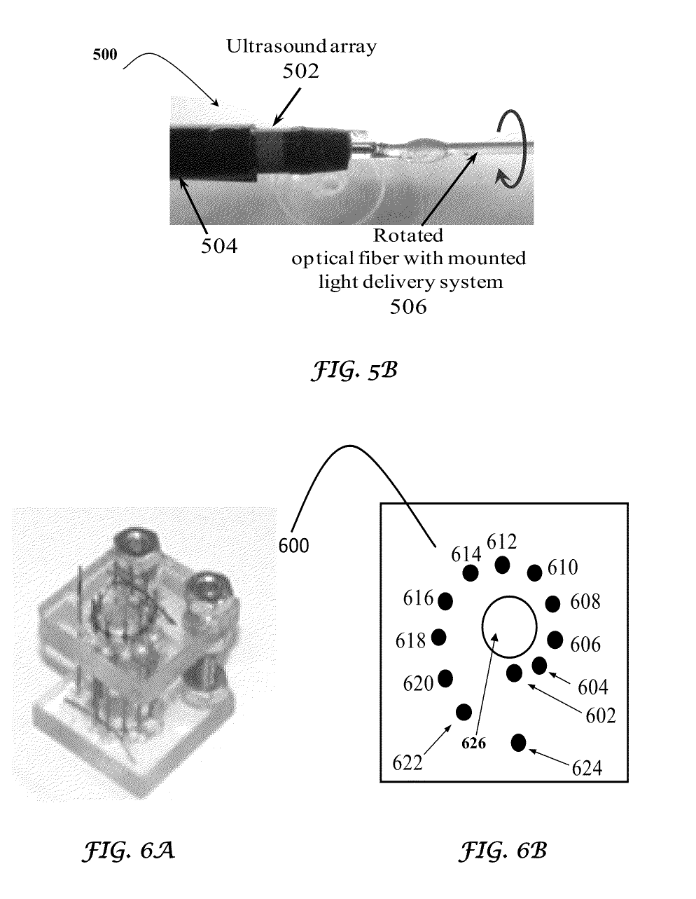

To test the invention, a point-target phantom 600 comprising of twelve graphite rods 602-624 was used. A photograph of a point-target phantom used in the studies and its structure are shown in FIGS. 6A and 6B, respectively. All twelve pencil rods 602-624 with diameter of 0.57±0.01 mm were oriented perpendicularly to an imaging cross-section 626. Eleven of them (602-622) were located spirally 4 to 9 mm away from the center of the phantom with 0.5 mm increment step. An inner diameter of a whole in the phantom is 6 mm. In addition, one rod 624 is located separately 10 mm away from the axis of the phantom.

A tissue-mimicking environment of the phantom to mimic artery's wall is not shown in FIGS. 6A and 6B was made out of 10% gelatin (Type A, Sigma-Aldrich, Inc.). Ultrasound properties were mimicked by silica particles (Sigma-Aldrich, Inc.) with 0.5% weight concentration and average size of 40 μm.⁴³ Optical scattering were mimicked by 20% of low fat milk (volume concentration).⁴⁴ The overall sizes of the phantom's body are measured to be 40×35×30 mm (L×W×H).

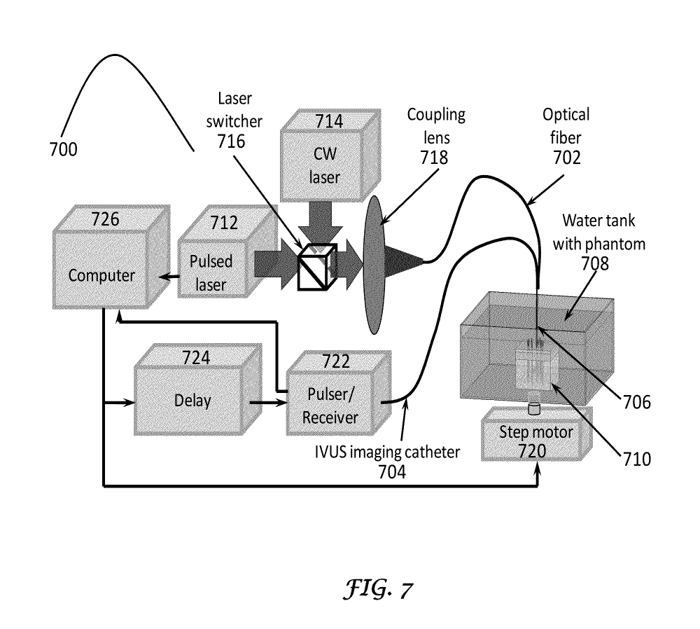

The block diagram of the IVUS/IVPA and elasticity imaging and therapy system 700 is shown in FIG. 7 with optical fiber 702, coupling lens 318, a CW laser 314 and laser switcher 716. The distal end of the integrated catheter 706 was inserted into the lumen and placed at the center of the vessel-mimicking phantom 710. The proximal ends of the light delivery system incorporated longitudinally with the IVUS imaging catheter 704 were connected with the output of the laser source 712 and the ultrasound pulser/receiver 722, respectively (FIG. 7). A tunable in near-infrared spectral region (680-960 nm and 1100-2400 nm) pulsed laser system 712 was used. Particularly, an OPO (Vibrant II, Opotek, Inc.) with pulse duration of 5 ns and repetition rate of 10 Hz was operated at 730 nm. To image phantom 710 without and with tissue-mimicking environment, the pulse energy measured on distal ends of both designs of light delivery systems comprised 1.4 mJ and 2.4 mJ respectively. The IVUS imaging catheter 704, used in both photoacoustic (echo only) and ultrasound (pulse-echo) modes, was operated by an ultrasound pulser/receiver 722 (5073PR, Panametrics-NDT, Inc.). Each radiofrequency (RF) signal consisted of photoacoustic and, delayed on 10 μs by a function generator 724 (33250A, Agilent, Inc.), ultrasound signatures. RF signals were captured by data acquisition card 726 (CompuScope 12200, GageScope, Inc.) and processed off-line.

The phantom was placed into a water tank 708 rotated precisely by a stepper motor 720 (ACCU Coder, Encoder Products, Inc.), while the integrated IVUS/IVPA imaging catheter 706 was fixed approximately on the axis of rotation to image point targets. One frame (360° rotation) included 251 A-lines. The averaging of 30 was applied to each A-line. RF signals were averaged, demodulated and scan converted to cover the 6.2-mm-radius field of view. No corrections or light fluency compensations were applied. Both designs of integrated IVUS/IVPA imaging catheters are capable of performing pullback 3-D imaging—a linear 1-D motion axis can be used to move the integrated catheter 706 relative to the phantom 710 thus allowing new cross-section to be imaged.

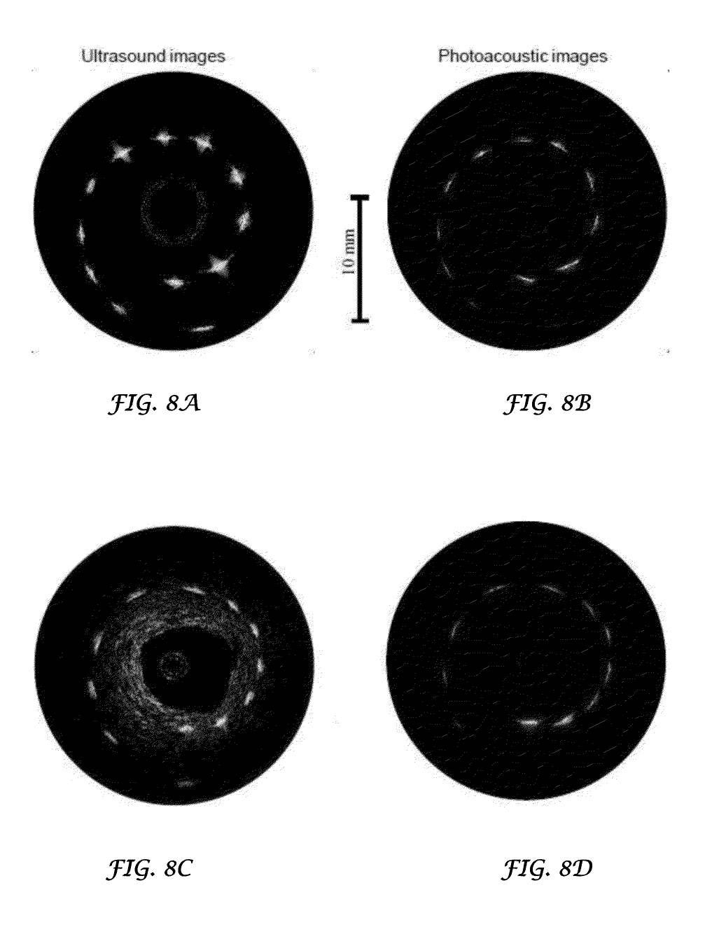

In order to demonstrate that the invention may be used for combined IVUS/IVPA imaging, the prototypes of integrated IVUS/IVPA imaging catheters shown in FIGS. 1B, 2A, 3A, and 5B were initially tested in phantom studies. The ultrasound and photoacoustic images of the point-target phantom obtained by the side fire fiber-based catheter are displayed in FIG. 8A and FIG. 8B, respectively. All ultrasound and photoacoustic images are shown in the dynamic range of 29 dB and 25 dB respectively. The ultrasound B-scan in FIG. 8A shows the structure of the phantom where all twelve point targets are visible. The brightness of the targets slightly decreases with the depth due to attenuation of the high-frequency (40 MHz) ultrasound in water and a divergence of the ultrasound beam.

The photoacoustic image in FIG. 8B demonstrates a decrease of the photoacoustic signal strength with the depth due to the light distribution in the phantom. Indeed, the light divergence increases the area of illumination with the distance from the catheter and, therefore, decreases the fraction of the light absorbed by the targets located further away from the catheter.

The ultrasound and photoacoustic B-scans of the point-target phantom within the tissue-mimicking environment are shown in FIGS. 8C and 8D, respectively. The ultrasound image in FIG. 8C exhibits the structure of the phantom. However, the decrease of the brightness of the targets with depth is greater than in FIG. 8A because ultrasound attenuation in tissue-mimicking environment is greater than that in water. This environment is not noticeable in the photoacoustic image in FIG. 8D due to modest light absorption in the gelatin and silica particles at 730 nm. However, the decrease of the photoacoustic transient magnitude from the targets in tissue-mimicking environment with the depth is greater than in water (FIG. 8B) because of the light scattering in the surrounding material. The light energy decays exponentially with the distance. The target that is further away from the catheter becomes invisible due to limited light energy reaches the target. Also, the generated photoacoustic wave attenuates as it travels to the transducer.

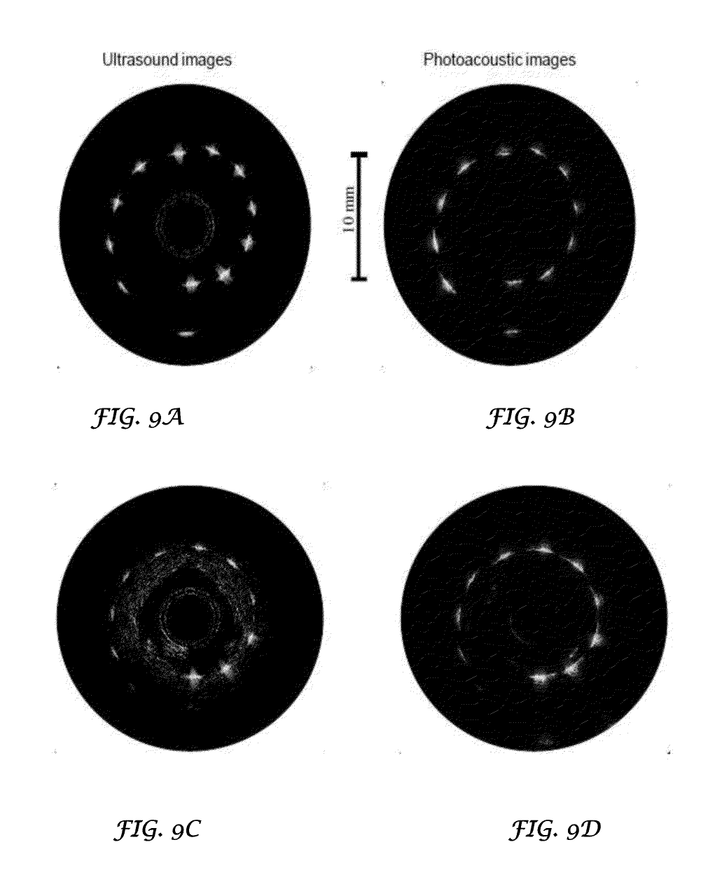

The ultrasound and photoacoustic B-scans of the point-target phantom obtained using the mirror-based catheter are shown in FIGS. 9A and 9B, respectively. All 12 targets are clearly indicated in FIG. 9A. Brightness of targets decreases slightly with depth.

The photoacoustic image in FIG. 9B indicates that the brightness of target points increases slightly for 1 through 7 targets (see FIG. 6B) because the closer point targets are not illuminated well while targets 8 through 11 were irradiated almost uniformly. Finally, the brightness of twelfth target is modest because this target is located too far from the catheter. There is a limited overlap between ultrasound and light beams.

The ultrasound and photoacoustic images of the point targets in the tissue-mimicking environment are shown in FIGS. 9C and 9D, respectively. Similar to FIG. 9A, all targets are detected in FIG. 9C and, as expected, the brightness of targets decreases with a distance from the transducer.

The photoacoustic image in FIG. 9D indicates that targets located closer to the catheter generate greater photoacoustic transients due to strong light scattering in the background. Indeed, the directivity of the laser beam is affected by light scattering, so the absorbed light energy rapidly decreases with depth.

Under clinical condition, light scattering in blood adds to that in soft tissues in the near-infrared spectral region⁴⁵. The whole blood in vessel was modeled by 20% solution of low-fat milk^{46,47}. The ultrasound and photoacoustic images of the phantom obtained by side fire fiber-based imaging catheter are shown in FIGS. 10A and 10B, respectively. As expected, FIG. 10A shows all 12 target points and the decrease rate of the brightness of the targets with the depth is almost the same as it is shown in FIG. 8A in the ultrasound image of the phantom in water. However, the light scattering results in a significant exponential attenuation in the milk solution so that the brightness of target points shown in FIG. 10B decreases rapidly with the depth. Nevertheless, the photoacoustic transients generated by 1 through 6 targets are clearly detectable. These targets are located 4 to 6.5 mm away from the imaging catheter so that photoacoustic imaging of the artery's walls in the presence of blood is possible. In addition, the brightness of the targets can be increased by an elevating the light energy as it will be discussed herein below.

Due to the contrast between optical absorption coefficients of blood⁴⁸ and aorta tissue⁴⁹, the photoacoustic signal from the blood may dominate in photoacoustic signature at certain wavelengths. To avoid the possible saturation of photoacoustic signal from blood, both side-fiber and mirror-based light delivery designs of the integrated catheters can be refocused several millimeters further from the catheter. The ultrasound and photoacoustic images of the phantom in tissue-mimicking environment obtained by refocused mirror-based catheter are shown in FIGS. 11A and 11B, respectively in dynamic ranges of 29 and 25 dB respectively. The transducer was shifted two millimeters away from the mirror. The ultrasound images in FIG. 11A and FIG. 9C differ due to different imaged cross-sections only. However, such refocusing degrades a photoacoustic transient from rods located closer while farer rods appear brighter (FIG. 11B).

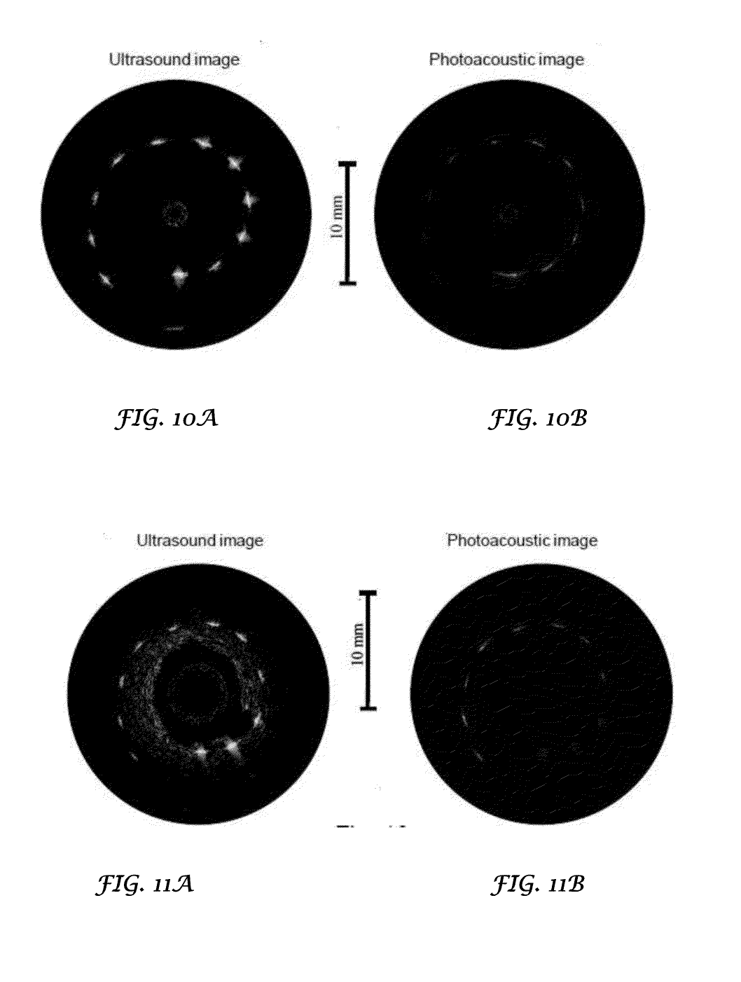

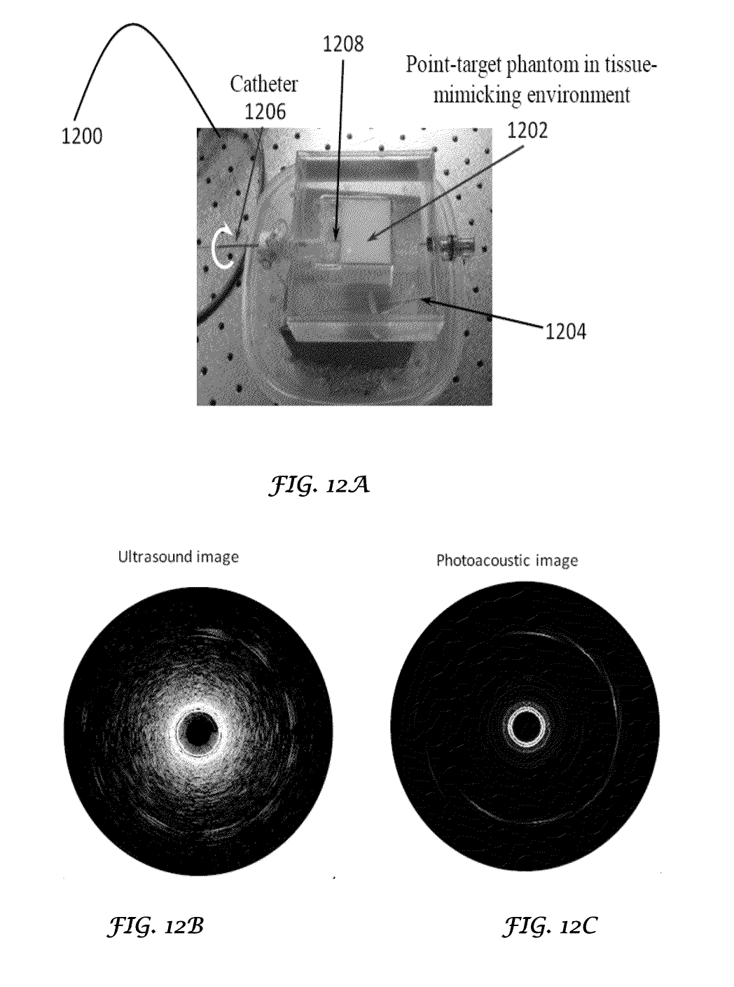

In order to demonstrate that the integrated IVUS/IVPA imaging catheter is capable of imaging blood vessels in vivo, the phantom shown in FIG. 6A was placed into tissue-mimicking environment and imaged using the catheter shown in FIG. 3A. FIG. 12A demonstrates the set-up 1200. The phantom 1202 was placed on one side of the water tank 1204 such that water filled the lumen 1208 where the catheter 1206 was inserted in. The ultrasound and photoacoustic images of the phantom are presented in FIGS. 12B and 12C, respectively. Comparing with results presented in FIGS. 8C and 8D, the geometry of the phantom is disfigured because of the catheter was not located on the axis of the phantom. However, the ultrasound image in FIG. 12B depicts clearly point targets and tissue-mimicking environment while the photoacoustic image in FIG. 12C identifies the areas with elevated optical absorption.

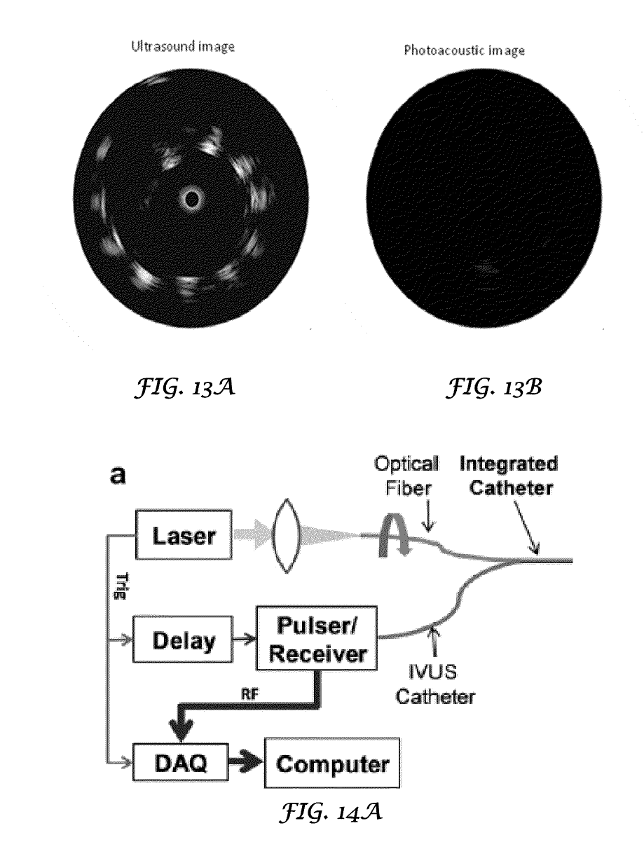

The ultrasound and photoacoustic B-scans of the point-target phantom obtained using the ultrasound array-based integrated IVUS/IVPA catheter with side fire fiber-based light delivery system are shown in FIGS. 13A and 13B, respectively. In this study the ultrasound array was stationary and ultrasound beam was electronically scanned. The optical fiber was also stationary directed and the rod 614 (see FIG. 6B). All 12 pencil rods are clearly indicated in FIG. 13A so the structure of the phantom is depicted clearly. Brightness of targets decreases slightly with depth but such significantly as in FIGS. 8A and 9A due to lower central frequency of the ultrasound array.

The photoacoustic image in FIG. 13B indicates the only one point source because the optical unit was not rotated. The mechanical rotation of the light delivery system is possible for sure as it is confirmed by results shown in FIGS. 12A and 12B.

While 2.5-mJ laser pulses were utilized for IVPA imaging of phantom placed in water, light absorption and light scattering in blood will attenuate the light energy thus making the photoacoustic imaging difficult. In addition, the optical absorption contrast between different tissue types may be limited⁴⁵. All of these require relatively high laser fluence output from the integrated catheter. The optical parts such as optical fibers and micro-mirrors used currently in the invention limit the laser energy so 14 mJ maximum can be delivered now into lumen. However, the construction of the invention itself does not limit the laser energy that could be potentially delivered is the appropriate optical parts with higher light damage thresholds will be utilized. For example, the commercially available micro-mirrors have a laser damage threshold of 1 J/cm² while that of the material of optical fiber's core used in the invention comprises over 30 GW/cm². Obviously, that the increase of light damage threshold of all optical parts will allow to increase the delivered light energy and, therefore, the optical contrast and imaging depth of IVUS imaging.

While the length of an integrated IVUS/IVPA imaging catheter does not typically exceed few meters, the light is will not be attenuated too much while propagating through the optical fiber with a regular light attenuation of several dB/km. However, the light delivery system mounted on distal tips of the fibers will cause the light lose. In the case of mirror-based system, the overall losses are estimated to be approximately 1.7% while the overall losses in side fire fiber-based system are expected to be around 6.5%. The anti-reflection coating can decrease the losses. In the case of side fire fiber-based system, it was assumed that the polishing angle β<β₀. Otherwise, if the light losses are increased up to 100% (see Eqs. (4) and (5)).

As shown in FIG. 5B an ultrasound array-based integrated IVUS/IVPA imaging catheter could not redirect light at an angle greater than 90° in spite of the fact that the effect of total internal reflection utilized. This aspect could limit applicability of such type of light delivery system in the array-based catheters. In such a case, the micro-optic-based system is preferable. As it is demonstrated in FIGS. 9A-9D, the mirror-based and, generally speaking, micro-optic-based light delivery system can be successfully in ultrasound array-based device. Note, the light scattering in blood and blood vessel tissues will result in a light redistribution so the side fire fiber-based light delivery system could also be capable of irradiating of imaging cross-section with lower efficiency.

The possible design of proximal ends of the integrated IVUS/IVPA imaging catheters includes the stepped motor or the like to rotate the catheter around its axis. The two wires are flattened and attached either to the fiber or to the fiber bundle whatever is used to deliver light. An ultrasound transducer is attached as it is shown in the figures, so that the cross-section of the combined catheter is circular along its working length. The optical fiber with wires should be coated, and be even along the fiber jacketing. The ready fiber is coupled with the laser and rotated by a motor.

The laser safety standards determined by ANSI limit the maximum acceptable radiation fluence on skin from 20 mJ/cm² in visible spectral range to 100 mJ/cm² at 1050 nm.⁵⁰ However, these values do not apply to blood and inner soft tissues. It was reported⁵¹ that the temperature increase in arterial tissues caused by laser pulse with fluence of 85 mJ/cm² comprises 5° C. and this value was considered as safe. The increase of blood temperature caused by a 2.4-mJ laser was estimated assuming the heat capacity and density of blood is equal to that of water. If the pulse is 2.7-times attenuated by blood at the distance equal to inversed extinction coefficient⁵² and size of the outlet of light delivery system is 1 mm×1 mm then the increase of blood temperature is approximately 10° C. This value can be, however, decreased with the greater size of outlet. Therefore, the IVPA imaging can be thermally safe.

### Example 2

The location and size of the necrotic lipid core is critical for analyzing the stability of atherosclerotic plaques (Falk 2006). Identification of vulnerable plaques depends on the distribution of lipid (Virmani 2011). Unfortunately, current invasive imaging modalities cannot reliably delineate spatially resolved lipid distribution (Vancraeynest et al. 2011). Intravascular optical coherence tomography (OCT) can detect lipid, but it lacks the imaging depth to assess the area of the lipid-rich plaques and requires temporal removal of luminal blood during imaging. Intravascular magnetic resonance imaging (MRI) has a better imaging depth, but it requires an occluding balloon to stabilize the catheter. Intravascular near-infrared spectroscopy (NIRS) can be performed in presence of blood, but the signals are not depth-resolved. Thus, a catheter-based imaging modality—ultrasound-guided intravascular photoacoustic imaging—was used to detect the depth-resolved distribution of lipid in atherosclerotic plaques in vivo.

Intravascular photoacoustic (IVPA) imaging is related to intravascular ultrasound (IVUS) imaging. In IVPA imaging, instead of sending acoustic waves into the tissue, a low energy, short laser pulse is emitted into the vessel wall. Upon absorption of the laser energy, thermal expansion of the tissue generates photoacoustic (PA) waves that are received by an ultrasound transducer. Among other factors, the amplitude of the PA signal is proportional to the optical absorption coefficient of the tissue (Oraevsky et al. 1997). Similar to IVUS imaging, the location of the absorber can be resolved based on the time of arrival of the PA signal. IVPA imaging is performed together with traditional IVUS imaging, which can delineate the anatomy of the vessel wall. Overall, the resolution of IVPA imaging is similar to IVUS imaging. For example, the axial resolution for IVPA imaging performed using a 40 MHz single element IVUS imaging catheter is tens of micrometers (Sethuraman et al. 2007). By scanning at multiple wavelengths, spectroscopic IVPA has been previously used to detect both endogenous and exogenous optical absorption contrast in arterial samples (Sethuraman et al. 2008; Allen and Beard 2009; Wang et al. 2009, 2010; Jansen et al. 2011).

Because of the abundance of C—H bonds in fatty acids, lipids absorbs strongly at around 1720 nm wavelength, around the first overtone of the C—H bond (Wang et al. 2002; Anderson et al. 2006). Therefore, IVPA imaging performed at 1720 nm wavelength can map the distribution of lipid deposits deep inside the plaques because imaging in this near infrared (NIR) wavelength range increases the imaging depth. Indeed, using excised samples of healthy and atherosclerotic rabbit aortas, 1720-nm IVPA imaging of lipid was successfully demonstrated ex vivo in the presence of luminal blood. In this example, an integrated IVPA/IVUS imaging catheter was used to detect, in vivo, lipid within a plaque in an atherosclerotic animal model using ultrasound-guided IVPA imaging.

Animal Model.

A Watanabe heritable hyperlipidemic (WHHL) rabbit was used as an animal model of atherosclerosis. WHHL rabbits have a high level of low-density lipoproteins (LDL) in their blood due to their genetic deficiency of LDL receptors, and can spontaneously develop atherosclerosis in the aorta and coronary arteries. Atherosclerotic plaques from WHHL rabbits are morphologically similar to human plaques (Masashi and Takashi 2009). In this study, an 18-month-old WHHL rabbit under normal diet was used for in vivo IVPA imaging. The rabbit weighted 3.4 kg at the time of the study.

Human Specimen.

A human right coronary artery (RCA) autopsy sample was acquired from the Department of Pathology at the University of Alabama at Birmingham. The sample was formalin-fixed and used for ex vivo ultrasound-guided IVPA imaging in a solution of human red blood cells (RBCs) acquired from the Blood Center of Central Texas. RBCs were diluted with saline to the concentration of RBCs in human whole blood.

IVPA/IVUS Imaging System and Catheter.

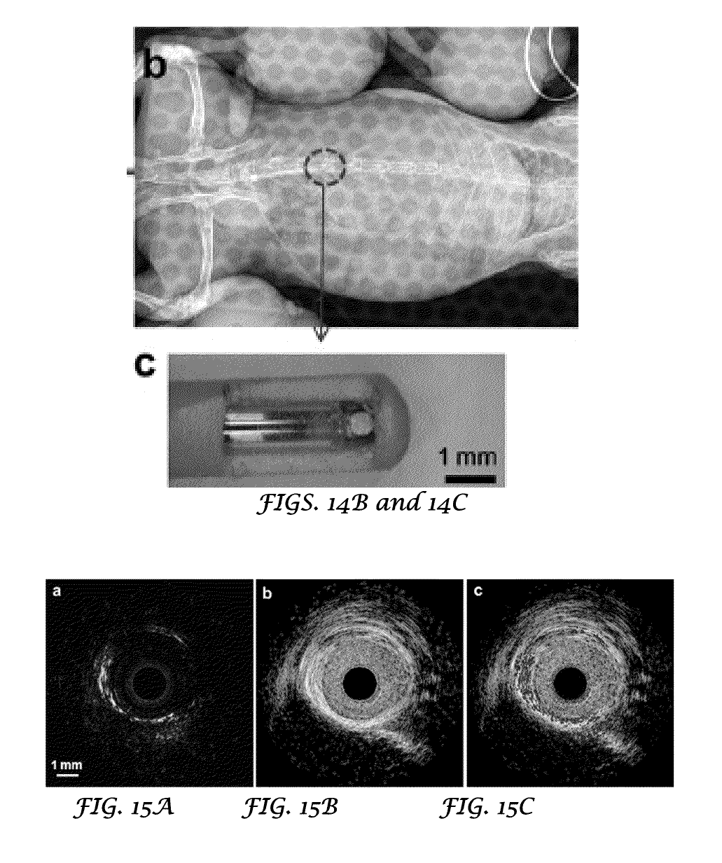

The combined IVPA and IVUS imaging system includes an IVUS imaging system interfaced with a pulsed laser source. The overall system was triggered by a tunable optical parametric oscillator (OPO) laser system (OPOTEK, Inc., Carlsbad, Calif. for in vivo imaging, and Spectra-Physics, Inc., Santa Clara, Calif. for ex vivo imaging) operating at a repetition rate of 10 Hz. Laser pulses with 3-5 ns duration were coupled with an integrated IVPA/IVUS imaging catheter. After each laser pulse, the data acquisition (DAQ) card (GaGe, Inc., Lockport, Ill.) started to sample the radiofrequency (RF) data at a 200 MHz sampling rate. Then, after a user-defined delay of 9 ms, conventional IVUS imaging was performed using the integrated catheter (FIG. 14A). Each scanning A-line included a spatially registered IVPA and IVUS signals from the vessel wall.

An integrated IVPA/IVUS imaging catheter was built with a single element 40 MHz IVUS imaging catheter (Boston Scientific, Inc., Natick, Mass.) and a light delivery system based on a side-fire fiber with a 600 mm core. By aligning the optical and acoustic beams, simultaneous IVPA/IVUS imaging could be performed in the overlapping region of the two beams (Karpiouk et al. 2010, 2012). Laser energy output from the integrated catheter was approximately 0.9 mJ/pulse. In this study, the total diameter of the distal end of the integrated catheter was around 2.2 mm (FIGS. 14B and 14C). Cross-sectional scans of the vessel wall were realized by rotating the catheter using a stepper motor with a gear system. Each co-registered IVPA and IVUS image included 256 scanning A-lines. No averaging was applied to IVPA or IVUS signals/images.

Image Acquisition.

Before in vivo imaging, 0.5 mL heparin (100 units/ml) was administrated to the anesthetized rabbit. The descending aorta of the WHHL rabbit was exposed via abdominal laparotomy. After isolating the descending aorta, a modified short 8F introducer was placed into the aorta distal to the renal arteries and advanced a short distance (˜2.5 cm). The artery distal to the introducer was tied off with a 4-0 silk ligature and secured with a 4-0 silk suture. Then, an integrated IVPA/IVUS imaging catheter was advanced into the introducer to perform ultrasound-guided IVPA imaging of the abdominal aorta. After in vivo imaging, the rabbit aorta was procured and imaged ex vivo in a cuvette filled with human RBCs solution within 24 h after the animal was sacrificed. The human RBCs solution was used to mimic whole blood during ex vivo imaging of rabbit aorta sample and human coronary artery sample. The protocol was approved by the Animal Welfare Committee of the University of Texas Houston Health Science Center.

Histopathology.

After ex vivo imaging of both artery samples, the imaged cross-sections were marked with a suture while the samples were still immersed within the blood cuvette. For the rabbit aorta, the marked cross-section was cut open using a scalpel blade and embedded in the tissue-freezing medium. The frozen tissue section was then sliced using a cryostat for Oil red O stain and hematoxylin-eosin (H&E) stain. The formalin fixed human sample was paraffin embedded and sliced with a microtome for H&E and CD68 stain, an immunohistochemistry marker for macrophages.

In Vivo IVPA Imaging.

A representative in vivo IVPA image (FIG. 15A) of the WHHL rabbit aorta shows the depth resolved distribution of lipid deposits inside the vessel wall. The spatially coregistered IVUS image (FIG. 15B) of the same cross-section clearly delineates the anatomy of the vessel wall. By combining the two images, the IVUS/IVPA image (FIG. 15C) shows that lipid deposits are located in the intimal layer of the aorta. IVPA signal generated from periadventitial fat can also be detected (FIG. 15C, 5 o'clock). The amplitude of backscattered IVUS signal from luminal blood was relatively strong compared with typical IVUS images (FIG. 15B). The stronger signal may be a result of reduced blood flow caused by tying off the distal end of the aorta. The background noise in the images was attributable to ambient electromagnetic noise from the surgical unit in the room where the in vivo study was performed. No significant motion artifacts caused by cardiac cycle were observed in FIG. 15A to 15C because the introducer was secured to the aorta and minimized the tissue motion.

Ex Vivo Imaging of Rabbit Aorta.

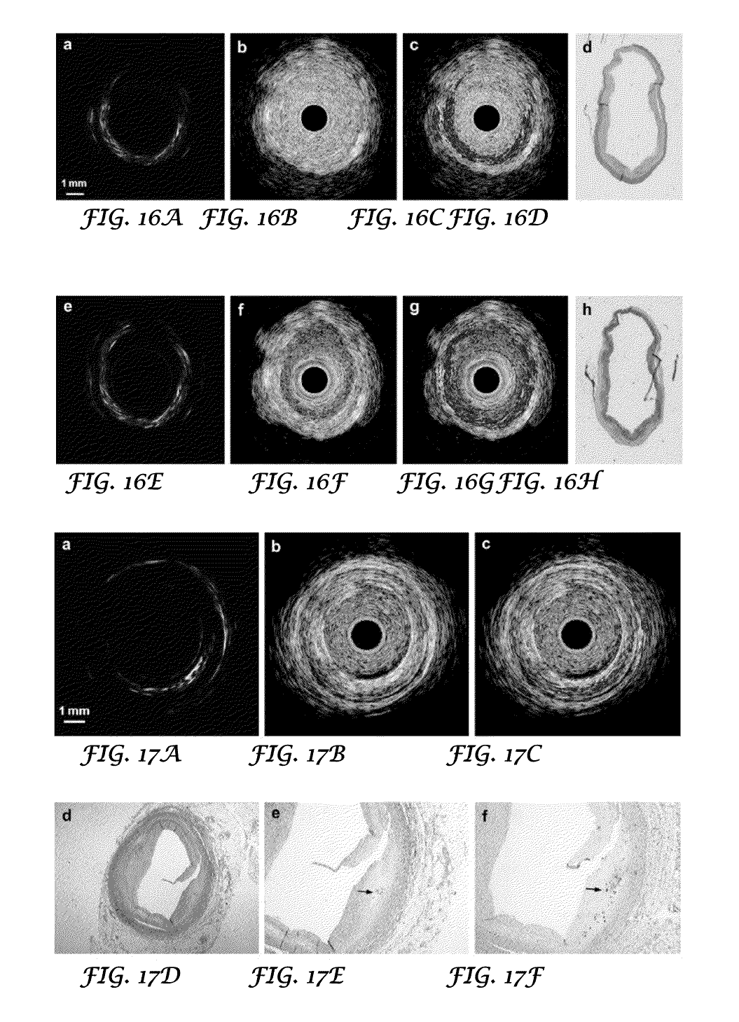

The excised rabbit aorta was immersed in human RBCs solution at the same concentration of RBCs in human whole blood for ultrasound-guided IVPA imaging at 1720 nm (FIG. 16A). The inner boundary of the aorta can barely be distinguished because of strong acoustic scattering from the stationary RBCs solution in the vessel lumen (FIG. 16B). The combined IVPA/IVUS image demonstrates the relative location of the IVPA signal source in the vessel wall (FIG. 16C). Then, the RBCs solution was diluted to approximately 10% of RBCs concentration in human whole blood. When imaged in the diluted RBCs solution, deeper imaging depth is achieved because of reduced light attenuation in luminal blood (FIG. 16E). In addition, because of lower acoustic scattering from luminal blood, the vessel wall can be more reliably identified in the IVUS image (FIG. 16F). The combined IVPA/IVUS image shows that lipid-rich plaques are present in the thickened intimal layer (FIG. 16G). H&E and Oil red O stain of the imaged cross-section confirmed that the vessel contained diffuse, lipid-rich plaques in the intimal layer of aorta (FIGS. 16D and 16H). Lipid at 12 o'clock region in FIGS. 16C and 16G were not visualized because of limited imaging depth of approximately 2.5 mm.

Ex Vivo Imaging of Lipid Core in Human RCA.

To demonstrate that ultrasound-guided IVPA can also detect lipid in human plaques, a human RCA sample was imaged in the RBCs solution at a concentration of human whole blood. Strong lipid signals are present from 3-6 o'clock in the IVPA image (FIG. 17A). The imaged artery has a thickened intimal layer (FIG. 17B). The hypo-echoic region from 4-6 o'clock next to the vessel lumen in the IVUS image (FIG. 17B) was caused by insufficient blood perfusion. The combined IVPA/IVUS image reveals the presence of lipid deposits located inside the intimal layer (FIG. 17C). Lipid from periadventitial fat also generated photoacoustic signal (FIG. 17C, 11—5 o'clock). H&E stain shows the vessel has mostly fibrous intima, and a lipid core containing early calcification (FIGS. 17D and 17E). CD68 stain for macrophages further confirmed inflammation in the lipid core (FIG. 17F).

Thus, it was found that combining IVPA imaging with IVUS imaging aids both imaging technologies and the distribution of lipid within atherosclerotic plaques (IVPA image) can be visualized within the context of the anatomical structure of the vessel (IVUS image). Furthermore, IVPA imaging shares similar data acquisition and signal processing procedures with IVUS imaging, which allows the integration of IVPA with IVUS imaging systems with minimal hardware changes. IVUS is already used in cardiac catheterization laboratories to guide interventions, and therefore, combined IVUS/IVPA imaging can be readily introduced to clinical practice. IVPA imaging depth inside the vessel wall is affected by the construction of the catheter and the distance between the lumen boundary and the catheter. Optical attenuation by the blood reduces the light energy reaching to the vessel wall. Imaging in the NIR wavelength range and the one-way transmission of light allows

IVPA to have a superior imaging depth in the presence of luminal blood compared with pure optical based imaging modalities, such as intravascular NIRS (Wang et al. 2002). Because the amplitude of PA signals relates to the local laser fluence but not the coherence of the laser beam inside the biologic tissue, removal of luminal blood, employed in intravascular OCT and intravascular MRI (Vancraeynest et al. 2011), is not needed. Using the current IVUS/IVPA integrated catheter, an imaging depth of approximately 2.5 mm was achieved in the presence of luminal blood (FIG. 15). Increasing the area of the overlapped region between the optical and acoustic beam can further improve the imaging depth. For example, deeper imaging can be achieved by adjusting the angle of the redirected laser beam relative to the acoustic beam of the imaging catheter (Karpiouk et al. 2010). Further miniaturization of the imaging catheter will result in an increase in the overlap between the acoustic and optical beams and, as a result, allow for improved imaging depth.

The size and flexibility of the integrated IVPA/IVUS imaging catheters are often not suitable to image coronary arteries. An integrated IVPA/IVUS catheter built with a 400 mm core side-fire fiber to a total diameter of 1.25 mm has been reported for ex vivo spectroscopic IVPA imaging in saline (Jansen et al. 2011). Because of the low laser energy required for lipid imaging at 1720 nm wavelength, an optical fiber with a smaller diameter can be used for the integrated catheter. In addition, the optical fiber should be incorporated into the driving cable of the catheter such that an integrated IVPA/IVUS imaging catheter will have a size similar to the clinically used IVUS imaging catheters.

The present study demonstrates that IVPA imaging can be used for in vivo depth resolved visualization and quantification of lipid in plaques. The in vivo experiments were performed using a WHHL rabbit model of atherosclerosis and the results were confirmed using ex vivo imaging of both atherosclerotic rabbit aorta and human coronary artery samples. Prior to imaging, the human sample used in this study was fixed in formalin at zero transmural pressure. Although the preservation procedure could potentially change the structure of the plaque and therefore affect the IVUS and IVPA images, the inventors observed an excellent correlation between the ultrasound-guided IVPA imaging and the histopathology results. Thus, a wide variety of human vessel samples with more diverse pathologies can be imaged, and a quantitative study can be performed to demonstrate the sensitivity and specificity of this technique.

Unlike previously proposed spectroscopic IVPA imaging of lipid around 1210 nm wavelength (Wang et al. 2010), where background PA signals were suppressed through spectroscopic (e.g., multi-wavelength) analysis, in this study a single wavelength of 1720 nm was used for lipid imaging. Because the PA signal from lipid was higher compared with other types of tissues, no multi-wavelength scan was required to further differentiate lipid-rich and background tissues. As a result, single-wavelength IVPA imaging reduces the cost of the system and increases the achievable imaging speed. In the current ultrasound guided IVPA imaging studies, a combined IVUS and IVPA image was acquired in about 30 s. The imaging speed was severely limited by the laser pulse repetition frequency of 10 Hz determined by the OPO laser system. It was found that for real-time imaging (30 frames/s), a laser with high, a few kHz, pulse repetition frequency is needed. Such high pulse repetition frequency may be achieved by several laser systems, (e.g., a diode laser, an OPO or dye laser driven by Q-switched diode-pumped solid-state laser, etc). Currently, Nd:YAGlasers operating at 1064 nm wavelength with 10 kHz frequency and several millijoules output energy are available. Therefore, real-time ultrasound-guided-IVPA imaging is possible.

Thus, the present invention includes a catheter-based ultrasound-guided IVPA imaging system was successfully tested for detection of lipid presence in vivo in an atherosclerotic rabbit aorta and ex vivo in a human RCA sample. The lipid-rich plaques and lipid core inside a diseased artery can be spatially resolved in the presence of luminal blood. This combined imaging method is a valuable asset for interventional cardiologists to guide percutaneous coronary intervention and can be used with or without the use of a contrast agent.

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. In embodiments of any of the compositions and methods provided herein, “comprising” may be replaced with “consisting essentially of” or “consisting of”. As used herein, the phrase “consisting essentially of” requires the specified integer(s) or steps as well as those that do not materially affect the character or function of the claimed invention. As used herein, the term “consisting” is used to indicate the presence of the recited integer (e.g., a feature, an element, a characteristic, a property, a method/process step or a limitation) or group of integers (e.g., feature(s), element(s), characteristic(s), propertie(s), method/process steps or limitation(s)) only.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

### REFERENCES

International Patent Application Publication No. WO/2010/080776: Systems and Methods for Making and Using Intravascular Ultrasound Systems with Photo-Acoustic Imaging Capabilities.
U.S. Pat. No. 7,711,413: Catheter Imaging Probe and Method.
C. L. de Korte, M. J. Sierevogel, F. Mastik, C. Strijder, J. A. Schaar, E. Velema, G. Pasterkamp, P. W. Serruys and A. F. W. van der Steen, Circulation, 105, 1627-1630 (2002). S. E. Nissen and P. Yock, Circulation, 103, 604-616 (2001).
S. P. Schwarzacher, P. J. Fitzgerald and P. G. Yock, Seminars in Interventional Cardiology 2, 1-9 (1997).
S. Sethuraman, S. R. Aglyamov, J. H. Amirian, R. Smalling and S. Y. Emelianov, IEEE Transactions on Ultrasonics, Ferroelectrics, and Frequency Control, 54, 978-986 (2007).
V. E. Gusev and A. A. Karabutov. Laser Optoacoustics, American Institute of Physics, New York; 1993.
C. K. N. Patel and A. C. Tam, Reviews of Modern Physics, 53, 517-550 (1981).
A. C. Tam, Reviews of Modern Physics, 58, 381-431 (1986).
L. Gao, K. J. Parker, R. M. Lerner and S. F. Levinson, Ultrasound in Medicine and Biology, 22, 959-977 (1996).
J. Ophir, S. K. Alam and B. S. Garra, Journal of Medical Ultrasonics, 29, 155-171 (2002).
J. Ophir, I. Cespedes, B. Gana, H. Ponnekanti, Y. Huang and N. Maklad, European Journal of Ultrasound, 3, 49-70 (1996).
K. J. Parker, L. Gao, R. M. Lerner and S. F. Levinson, IEEE Engineering in Medicine and Biology Magazine, 15, 52-59 (1996).
J. D'Hooge, A. Heimdal, F. Jamal and e. all, European Journal of Echocardiography, 1, 154-170 (2000).
C. L. de Korte, E. I. Cespedes, A. F. W. van der Steen, G. Pasterkamp and N. Bom, European Journal of Ultrasound, 7, 219-224 (1998).
K. W. Hollman, S. Y. Emelianov, J. H. Neiss and e. al., Cornea, 21, 68-73 (2002).
S. F. Levinson, M. Shinagawa and T. Sato, Journal of Biomechanics, 28, 1145-1154 (1995).
B. M. Shapo, J. R. Crowe, A. R. Skovoroda, K. J. Eberle, N. A. Cohn and M.
O'Donnell, IEEE Transactions on Ultrasonics, Ferroelectrics, and Frequency Control, 43, 234-246 (1996).
J. Shah, S. R. Aglyamov, K. Sokolov, T. E. Milner and S. Y. Emelianov, Optics Express, 16, 3776-3785 (2008).
J. Shah, S. Park, S. r. Aglyamov, T. Larson, L. Ma, K. Sokolov, K. Johnston, Milner
T. and S. Y. Emelianov, Proceedings of the 2008 SPIE Photonics West Symposium: Photons Plus Ultrasound Imaging and Sensing, 6856, 68560U:1-7 (2008).
J. Shah, S. Park, S. R. Aglyamov, T. Larson, L. Ma, K. Sokolov, K. Johnston, T. E. Milner and S. Y. Emelianov, Journal of Biomedical Optics, 13, 034024 (2008).
J. Shah, S. Thompson, T. E. Milner and S. Y. Emelianov, Lasers in Surgery and Medicine 40, 680-687 (2008).
X. Jin, Y. Xu, L. V. Wang, Y. R. Fang, C. I. Zanelli and S. M. Howard, Medical Physics, 32, 5-11 (2004).
T. Long, V. Amin, S. McClure, R. Roberts, L. Wu, M. Heise and T. Ryken, Proceedings of the 2008 SPIE Photonics West Symposium: Photons Plus Ultrasound: Imaging and Sensing, 6513, (2007).
U. Utzinger and R. Richards-Kortum, Journal of Biomedical Optics, 8, 121-147 (2003).
S. R. Aglyamov, A. R. Skovoroda, H. Xie, K. Kim, J. M. Rubin, M. O'Donnell, T. W. Wakefield, D. Myers and S. Y. Emelianov, International Journal of Biomedical Imaging, 2007, 11 pages (2007).
J. Shah, A. B. Karpiouk and S. Y. Emelianov, Abstract and Presentation at the 2009 SPIE Photonics West Symposium: Photons Plus Ultrasound: Imaging and Sensing, (2009).
J. Shah, J. Mendeloff and S. Y. Emelianov, Abstract of the 25th Annual Houston Conference on Biomedical Engineering Research, The Houston Society for Engineering in Medicine and Biology, 53 (2008).
R. Virmani, F. D. Kolodgie, A. P. Burke, A. Farb and S. M. Schwartz, Arteriosclerosis, Thrombosis, and Vascular Biology, 20, 1262-1275 (2000).
M. Naghavi, P. Libby, E. Falk, S. W. Casscells, S. Litovsky, J. Rumberger, J. J. Badimon, C. Stefanadis, P. Moreno, G. Pasterkamp, Z. Fayad, P. H. Stone, S. Waxman, P. Raggi, M. Madjid, A. Zarrabi, A. Burke, C. Yuan, P. J. Fitzgerald, D. S. Siscovick, C. L. de Korte, M. Aikawa, K. E. J. Airaksinen, G. Assmann, C. R. Becker, J. H. Chesebro, A. Farb, Z. S. Galis, C. Jackson, I.-K. Jong, W. Koenig, R. A. Lodder, K. March, J. Demirovic, M. Navab, S. G. Priori, M. D. Rekhter, R. Bahr, S. M. Grundy, R. Mehran, A. Colombo, E. Boerwinkle, C. Ballantyne, J. Insull, W., R. S. Schwartz, R. Vogel, P. W. Serruys, G. K. Hansson, D. P. Faxon, S. Kaul, H. Drexler, P. Greenland, J. E. Muller, R. Virmani, P. M. Ridker, D. P. Zipes, P. K. Shah and J. T. Willerson, Circulation, 108, 1664-1672 (2003).
H. C. Stary, A. B. Chandler and R. E. Dinsmore, Arteriosclerosis, Thrombosis, and Vascular Biology 15, 1512-1531 (1995).
P. C. Beard and T. N. Mills, Physics in Medicine and Biology, 42, 177-198 (1997).
A. Berlis, H. Lutsep, S. Barnwell, A. Norbash, L. Wechsler, C. A. Jungreis, A. Woolfenden, G. Redekop, M. Hartmann and M. Schumacher, Stroke, 35, 1112-1116 (2004).

P. M. Henrichs, J. W. Meador, J. M. Fuqua and A. A. Oraevsky, Proceedings of SPIE, 5697, 217-223 (2005).
Y. Y. Petrov, I. Y. Petrova, I. A. Patrikeev, R. O. Esenaliev and D. S. Prough, Optics Letters, 31, 1827-1829 (2006).
M. C. Pilatou, N. J. Voogd, F. F. M. de Mul, W. Steenbergena and L. N. A. van Adrichem, Review of Scientific Instruments, 74, 4495-4499 (2003).
H. F. Zhang, K. Maslov, M. Sivaramakrishnan, G. Stoica and L. V. Wang, Applied Physics Letters, 90, 053901 (2007).
S. Sethuraman, J. H. Amirian, S. H. Litovsky, R. Smalling and S. Y. Emelianov, Optics Express, 15, 16657-16666 (2007).
S. Sethuraman, J. H. Amirian, S. H. Litovsky, R. Smalling and S. Y. Emelianov, Optics Express, 16, 3362-3367 (2008).
Boston Scientific Inc. Atlantis RS Pro imaging Catheter. See at http://www.bostonscientific.com
Volcano Corporation Inc. Visions® PV8.2F imaging catheter. See at http://www.volcanocorp.com.
P. C. Beard, F. Perennes, E. Draguioti and T. N. Mills, Optics letters, 23, 1235-1237 (1998).
P. A. Fomitchov, A. K. Kromine and S. Krishnaswamy, Applied Optics, 41, 4451-4459 (2002).
J. G. Fujimoto, S. A. Boppart, G. J. Tearney, B. E. Bouma, C. Pitris and M. E. Brezinski, Heart, 82, 128-133 (1999).
E. L. Madsen, J. A. Zagrebski, M. C. MacDonald and G. R. Frank, American Association of Physical Medicine, 18, 1171-1181 (1991).
M. D. Waterworth, B. J. Tarte, A. J. Joblin, T. van Doom and H. E. Niesler, Australasian Physical and Engineering Sciences in Medicine 18, 39-44 (1995).
W.-F. Cheong, S. A. Prahl and A. J. Welch, IEEE Journal of Quantum Electronics, 26, 2166-2185 (1990).
G. Mitic, J. Kolzel, J. Otto, E. Plies, G. Solkner and W. Zinth, Applied Optics, 33, 6699-6710 (1994).
B. W. Pogue and M. S. Patterson, Journal of Biomedical Optics, 11, 041102 (2006).
S. Prahl, Oregon Medical Laser Center, http://omlc.ogi.edu/spectra/hemoglobin/summary.html, (2000).
J. M. C. van Gernert, R. M. Verdaasdonk, E. G. Stassen and G. Schets, Lasers in Surgery and Medicine, 5, 235-262 (1985).
ANSI. American national standards for Safe Use of Lasers. Volume Z136.1-2000. New York, USA: American National Standards Institute; 2000.
S. Sethuraman, S. R. Aglyamiv, R. W. Smalling and S. Y. Emelianov, Ultrasound in Medicine and Biology, 34, 299-308 (2008).
D. J. Faber, M. C. G. Aalders, E. G. Mik, B. A. Hooper, M. J. C. van Gernert and T. G. van Leeuwen, Physical Review Letters, 93, 028102 (2004).
Allen T, Beard P C. Photoacoustic characterization of vascular tissue at NIR wavelengths. Proceeding of SPIE 2009; 7177:71770 A 1-9.
Anderson R R, Farinelli W, Laubach H, Manstein D, Yaroslaysky A N, Gubeli J 3rd, Jordan K, Neil G R, Shinn M, Chandler W. Selective photothermolysis of lipid-rich tissues: A free electron laser study. Lasers Surg Med 2006; 38:913-919.
Falk E. Pathogenesis of Atherosclerosis. J Am Coll Cardiol 2006; 47: C7-C12.
Jansen K, van der Steen A F W, van Beusekom H M M, Oosterhuis J W, van Soest G. Intravascular photoacoustic imaging of human coronary atherosclerosis. Opt Lett 2011; 36:597-599.
Karpiouk A B, Wang B, Amirian J, Smalling R W, Emelianov S Y. Feasibility of in vivo intravascular photoacoustic imaging using integrated ultrasound and photoacoustic imaging catheter. J Biomed Opt 2012 (in press). doi:10.1117/1.JB0.17.9.096008.
Karpiouk A B, Wang B, Emelianov S Y. Development of a catheter for combined intravascular ultrasound and photoacoustic imaging. Rev Sci Instrum 2010; 81:014901.
Masashi S, Takashi I. The Watanabe heritable hyperlipidemic (WHHL) rabbit, its characteristics and history of development: A tribute to the late Dr. Yoshio Watanabe. Atherosclerosis 2009; 207:1-7.
Oraevsky A A, Jacques S L, Tittel F K. Measurement of tissue optical properties by time-resolved detection of laser-induced transient stress. Appl Opt 1997; 36:402-415.
Sethuraman S, Aglyamov S R, Amirian J H, Smalling R W, Emelianov S Y. Intravascular Photoacoustic Imaging Using an IVUS Imaging Catheter. IEEE Trans Ultrason Ferroelectr Freq Control 2007; 54:978-986.
Sethuraman S, Amirian J H, Litovsky S H, Smalling R W, Emelianov S Y. Spectroscopic intravascular photoacoustic imaging to differentiate atherosclerotic plaques. Optics Express 2008; 16:3362-3367.
Vancraeynest D, Pasquet A, Roelants V, Gerber B L, Vanoverschelde J-L J. Imaging the Vulnerable Plaque. JAm Coll Cardiol 2011; 57:1961-1979. Virmani R. Are our tools for the identification of TCFA ready and do we know them? JAm Coll Cardiol 1 mg 2011; 4:656-658.
Wang B, Karpiouk A, Yeager D, Amirian J, Litovsky S, Smalling R, Emelianov S. Intravascular photoacoustic imaging of lipid in atherosclerotic plaques in the presence of luminal blood. Opt Lett 2012; 37: 1244-1246.
Wang B, Su J L, Amirian J, Litovsky S H, Smalling R, Emelianov S. Detection of lipid in atherosclerotic vessels using ultrasound-guided spectroscopic intravascular photoacoustic imaging. Opt Express 2010; 18:4889-4897.
Wang B, Yantsen E, Larson T, Karpiouk A B, Sethuraman S, Su J L, Sokolov K, Emelianov S Y. Plasmonic intravascular photoacoustic imaging for detection of macrophages in atherosclerotic plaques. Nano Lett 2009; 9:2212-2217.
Wang J, Geng Y-J, Guo B, Klima T, Lal B N, Willerson J T, Casscells W. Near-infrared spectroscopic characterization of human advanced atherosclerotic plaques. J Am Coll Cardiol 2002; 39:1305-1313.

What is claimed is:

1. A method of imaging and treating a target tissue without the need to occlude or dilute luminal blood in a subject comprising the steps of:
identifying a subject in need of treatment of a target tissue using an intravascular imaging and therapeutic device capable of combined intravascular ultrasound and photoacoustic imaging;
irradiating the target tissue with electromagnetic radiation at a single wavelength from an intravascular imaging and therapeutic device comprising:
a catheter with a proximal end and a distal end;
one or more intravascular ultrasound units comprising a proximal end and a distal end, wherein the distal end comprises one or more single-element ultrasound transducer elements at a proximal end;
one or more optical units comprising a proximal end and a distal end combination, wherein the distal end comprises at least one of one or more optical fibers or one or more optical bundles, wherein the proximal end comprises a port to couple at least one optical unit to a light source that irradiates at a single wavelength;
an imager connected to the proximal end of the unit to capture one or more ultrasound and optical images; and
performing at least one of an imaging or a therapy of the target tissue.

2. The method of claim 1, wherein single wavelength is selected from one of 1060, 1064, 1070, 1200, 1210, 1700, 1710, 1715, 1720, 1725, 1730, or 1740 nm.

3. The method of claim 1, the distribution of the ultrasound impedance is reconstructed by transmitting of short ultrasound waves into the target tissue with consequent detection of at least one of reflected or scattered ultrasound waves.

4. The method of claim 1, wherein the distribution of the optical absorption is reconstructed by transmitting of short light pulses of the same wavelength into the target tissue with a consequent detection of ultrasound waves generated in the tissue due to thermal expansion of the tissue due to absorbed light energy.

5. The method of claim 1, wherein the one or more ultrasound units irradiate an artery with long ultrasound pulses to perform an acoustic therapy of the artery.

6. The method of claim 1, wherein the one or more optical units can irradiate tissues by CW or long-pulse light to perform an optical therapy.

7. The method of claim 1, wherein the at least one of optical therapy, acoustic therapy, tissue target imaging, or therapy and imaging can be performed either simultaneously or separately.

8. The method of claim 1, wherein the imager is capable of providing imaging results or therapy results in a format determined by a user.

9. The method of claim 1, wherein the imager has a penetration depth of 1 to 5 mm.

10. The method of claim 1, wherein the imager has a resolution of 50 micrometers.

11. The method of claim 1, wherein the imager is able to detect the presence and spatially resolved location of lipid in the arterial wall.

12. The method of claim 1, wherein the imager modifies the contrast between lipid tissue and water-based tissue by increasing the temperature at the target tissue, wherein photoacoustic signal intensity of the lipid based tissue decreases and the water-based tissue increases.

13. The method of claim 1, further comprising the step of mechanically or electrically steering an ultrasound beam to generate a two-dimensional cross-sectional image of the target.

14. The method of claim 1, further comprising the step of using a contrast agent in the target tissue.

15. A method of imaging and treating a target tissue in vivo without the need to occlude or dilute luminal blood in a subject comprising the steps of:
identifying a subject in need of treatment of a target tissue using an intravascular imaging and therapeutic device capable of combined intravascular ultrasound and photoacoustic imaging;
irradiating the target tissue with electromagnetic radiation at a single wavelength selected from wavelengths in which lipid and water have a different index of refraction comprising:
a catheter with a proximal end and a distal end;
one or more intravascular ultrasound units comprising a proximal end and a distal end, wherein the distal end comprises one or more single-element ultrasound transducer elements at a proximal end;
one or more optical units comprising a proximal end and a distal end combination, wherein the distal end comprises at least one of one or more optical fibers or one or more optical bundles, wherein the proximal end comprises a port to couple at least one optical unit to a light source that irradiates at a single wavelength;
an imager connected to the proximal end of the unit to capture one or more ultrasound and optical images; and
performing at least one of an imaging or a therapy of the target tissue in vivo.

16. The method of claim 15, wherein single wavelength is selected from one of 1060, 1064, 1070, 1200, 1210, 1700, 1710, 1715, 1720, 1725, 1730, or 1740 nm.

17. The method of claim 15, the distribution of the ultrasound impedance is reconstructed by transmitting of short ultrasound waves into the target tissue with consequent detection of at least one of reflected or scattered ultrasound waves.

18. The method of claim 15, wherein the distribution of the optical absorption is reconstructed by transmitting of short light pulses of the same wavelength into the target tissue with a consequent detection of ultrasound waves generated in the tissue due to thermal expansion of the tissue due to absorbed light energy.

19. The method of claim 15, wherein the one or more ultrasound units irradiate an artery with long ultrasound pulses to perform an acoustic therapy of the artery.

20. The method of claim 15, wherein the one or more optical units can irradiate tissues by CW or long-pulse light to perform an optical therapy.

21. The method of claim 15, wherein the at least one of optical therapy, acoustic therapy, tissue target imaging, or therapy and imaging can be performed either simultaneously or separately.

22. The method of claim 15, wherein the imager is capable of providing imaging results or therapy results in a format determined by a user.

23. The method of claim 15, wherein the imager has a penetration depth of 1 to 5 mm.

24. The method of claim 15, wherein the imager has a resolution of 50 micrometers.

25. The method of claim 15, wherein the imager is able to detect the presence and spatially resolved location of lipid in the arterial wall.

26. The method of claim 15, wherein the imager modifies the contrast between lipid tissue and water-based tissue by increasing the temperature at the target tissue, wherein photoacoustic signal intensity of the lipid based tissue decreases and the water-based tissue increases.

27. The method of claim 15, further comprising the step of mechanically or electrically steering an ultrasound beam to generate a two-dimensional cross-sectional image of the target.

28. The method of claim 15, further comprising the step of using a contrast agent in the target tissue.
